(19) United States
(12) Patent Application Publication
(10) Pub. No.: US 2023/0348980 A1
(43) Pub. Date: Nov. 2, 2023

(54) SYSTEMS AND METHODS OF DETECTING A RISK OF ALZHEIMER'S DISEASE USING A CIRCULATING-FREE MRNA PROFILING ASSAY

(71) Applicant: Molecular Stethoscope, Inc., South San Francisco, CA (US)

(72) Inventors: Michael NERENBERG, South San Francisco, CA (US); Arkaitz IBARRA, South San Francisco, CA (US); Jiali ZHUANG, South San Francisco, CA (US); Alexander ACOSTA, South San Francisco, CA (US)

(21) Appl. No.: 17/932,244

(22) Filed: Sep. 14, 2022

Related U.S. Application Data
(63) Continuation of application No. PCT/US2021/023015, filed on Mar. 18, 2021.
(60) Provisional application No. 62/991,513, filed on Mar. 18, 2020.
(60) Provisional application No. 62/992,723, filed on Mar. 20, 2020.

(51) Int. Cl. C12Q 1/6883; G16B 30/20; G16B 40/10

(52) U.S. Cl. CPC C12Q 1/6883; G16B 30/20; G16B 40/10; C12Q 2600/158; C12Q 2600/118

(57) ABSTRACT

Disclosed herein are panels related to the diagnosis of diseased tissue in a subject. The disclosed panels and related methods are used to predict or assess whether a subject has a neurodegenerative disorder taking into account the age of the subject. Some embodiments of the methods include applying a gene filter based on the age of the subject and generating an output of gene expression data which takes into account differences in gene profiles seen in tissues as they age.

### CROSS-REFERENCE

This application is a Continuation Application of International Patent Application PCT/US2021/023015, filed Mar. 18, 2021, which claims priority to U.S. Provisional Patent Application Ser. No. 62/991,513, filed Mar. 18, 2020, and U.S. Provisional Patent Application Ser. No. 62/992,723, filed Mar. 20, 2020, each of which is incorporated herein by reference herein in its entirety.

### BACKGROUND

Alzheimer's disease (AD) is a neurodegenerative disorder marked by cognitive and behavioral impairment that significantly interferes with patients' normal day-to-day function. It is an incurable disease with a long preclinical period and progressive course.

Alzheimer's disease is the most common cause of dementia affecting a large portion of the elderly population globally and it is projected to triple by 2050. Alzheimer's disease is a neurodegenerative condition generally characterized by the accumulation of amyloid-β peptide, deposition of tau proteins and neurofibrillary tangles, onset of synaptic and neuronal dysfunction, activation of inflammatory response caused by microglia, and mitochondria dysfunction. The current diagnostic guidelines of preclinical Alzheimer's disease utilize psychometric tests for establishing the existence of cognitive impairment and subsequently use imaging and cerebrospinal fluid (CSF) biomarkers to determine whether the impairment is caused by Alzheimer's disease. Although post-mortem histology remains the gold standard for establishing Alzheimer's disease pathology, assessment of CSF Aβ1-42 and amyloid positron-emission tomography (PET) can be used as surrogates. Furthermore, changes in the brain manifest years before clinical symptoms with known pre-symptomatic changes including cortical thinning and deposition of amyloid-β, tau proteins, and neurofibrillary tangles. While these pathological changes can be measured by imaging tests and CSF protein markers, imaging modalities are costly and CSF collection is invasive. Therefore, there is a need for highly accessible non-invasive tests for Alzheimer's disease diagnosis.

### SUMMARY

Disclosed herein is a method of detecting Alzheimer's disease (AD) in a subject, the method comprising: (a) quantifying cell-free messenger RNA (cf-mRNA) levels of a plurality of cf-mRNAs in a biological sample; and (b) processing one or more of said levels of said plurality of cf-mRNAs to identify a disease state of a tissue of the subject and an age of the subject, wherein processing comprises comparing the cf-mRNA levels in the subject to a threshold value of the plurality of cf-mRNAs. The biological sample can comprise blood of the subject. Processing can comprise applying a machine learning classifier to the one or more of the levels of said plurality of cf-mRNAs. The machine learning classifier can comprise a LASSO regression model. The method can further comprise (c) quantifying cf-mRNA levels of the plurality of cf-mRNAs in a second biological sample and (d) processing one or more of said levels of the plurality of cf-mRNAs in the second biological sample to identify a second disease state of said tissue of said subject. The second biological sample can be obtained after the subject has received a treatment or therapy for a neurodegenerative disorder. The treatment or therapy can comprise one or more of a cholinesterase inhibitors or memantine. The quantifying can comprise subjecting the plurality of cf-mRNAs to at least one of reverse transcription, polynucleotide amplification, sequencing, probe hybridization, microarray hybridization, or a combination thereof.

The method can further comprise forming a next-generation sequencing (NGS) library comprising a plurality of cDNAs derived from the plurality of cf-mRNAs. The quantifying can further comprise detecting a proportion of the plurality of cf-mRNA that contributes to the biological sample not from blood. The quantifying can further comprise detecting a proportion of the plurality of cf-mRNAs that contributes to the biological sample from the subject's brain. The plurality of cf-mRNAs can correspond to two or more genes selected from the group consisting of KIAA0100, MAGl1, NNMT, MXD1, ZNF75A, SELL, ASS1, MNDA, and AC132217.4. The method can further comprise identifying the subject as having a high risk of Alzheimer's disease and recommending a treatment. The method can further comprise treating the patient for Alzheimer's disease. The treatment can comprise one or more of a cholinesterase inhibitor or memantine.

Disclosed herein is a method of detecting a stage of Alzheimer's disease (AD) in a subject, the method comprising: (a) obtaining a biological sample from the subject; and (b) detecting cell-free messenger RNA (cf-mRNA) levels of a plurality of cf-mRNAs in the biological sample, wherein the plurality of cf-mRNAs correspond to two or more genes selected from the group consisting of KIAA0100, MAGl1, NNMT, MXD1, ZNF75A, SELL, ASS1, MNDA, and AC132217.4. The method can further comprise processing the levels of the plurality of cf-mRNAs using a machine learning classifier. The machine learning classifier can comprise a LASSO regression model. The method can further comprise (c) obtaining a second biological sample from the subject; and (d) detecting cell-free messenger RNA (cf-mRNA) levels of a plurality of cf-mRNAs in the second biological sample. The second biological sample can be obtained after the subject has received a treatment or therapy for a neurodegenerative disorder. The treatment or therapy can comprise one or more of a cholinesterase inhibitors or memantine. The method can further comprise identifying a risk of the subject for having a stage of Alzheimer's disease. The stage of Alzheimer's disease can be selected from: preclinical Alzheimer's disease, mild cognitive impairment due to Alzheimer's disease, mild dementia due to Alzheimer's disease, moderate dementia due to Alzheimer's disease, or severe dementia due to Alzheimer's disease. The method can further comprise comparing the cf-mRNA levels of the plurality of cf-mRNAs to a threshold value of cf-mRNA levels of the plurality of cf-mRNAs.

The method can further comprise inputting the cf-mRNA levels to a classifier to obtain a risk score, wherein the risk score is indicative of a likelihood that the subject has AD. The classifier can be a trained machine learning algorithm. The trained machine learning algorithm can comprise a LASSO regression model. The trained machine learning algorithm can be trained using biological samples from subjects diagnosed with Alzheimer's disease. The risk score can be determined a sensitivity of at least 80%. The risk score can be determined a sensitivity of at least 90%. The risk score can have a cutoff value of 0.44. The risk score can indicate a particular development status of Alzheimer's disease for the subject. Prior to determining the risk score of the subject, the subject may not have been diagnosed with Alzheimer's disease. The method can further comprise generating a report based on the risk score. The method can further comprise transmitting the report to a health practitioner. The report can comprise a recommendation for administering cholinesterase inhibitors and/or memantine.

The method can further comprise assigning a clinical dementia rating (CDR) score or a mini-mental state examination (MMSE) score to the subject. The assigning can further comprise (a) quantifying cf-mRNA levels of a second plurality of cf-mRNAs in the biological sample, wherein the second plurality of cf-mRNAs corresponds to two or more genes selected from the group consisting of SLU7, HNRNPA2B1, GGCT, NDUFA12, HSPB11, ATP6V1B2, SASS6, SUMO1, KRCC1, and LSM6; and (b) comparing the second plurality of cf-mRNA levels in the subject to a threshold value of the second plurality of cf-mRNAs. The quantifying can comprise subjecting the second plurality of cf-mRNAs to at least one of reverse transcription, polynucleotide amplification, sequencing, probe hybridization, microarray hybridization, or a combination thereof. The biological sample can be plasma or serum. The biological sample can be cerebrospinal fluid. The first plurality of cf-mRNAs and the second plurality of cf-mRNAs can be from at least two of cerebrum, cerebellum, dorsal root ganglion, superior cervical ganglion, pineal gland, amygdala, trigeminal ganglion, cerebral cortex, and hypothalamus. The method can further comprise monitoring AD progression. The monitoring can comprise a magnetic resonance imaging (MRI) brain scan or computed tomography (CT) brain scan. The method can further comprise administering a mental acuity test to the subject.

Disclosed herein is a method of detecting Alzheimer's disease (AD) in a subject, the method comprising: (a) quantifying cell-free messenger RNA (cf-mRNA) levels of a plurality of cf-mRNAs in a biological sample, wherein the plurality of cell-free mRNA corresponds to genes encoding transcriptional factors involved in at least one of the sirtuin signaling pathway, IL-8 signaling pathway, protein ubiquitination pathway, oxidative phosphorylation pathway, sumoylation pathway, mitochondrial dysfunction pathway, inflammasome pathway, GABA receptor signaling pathway, netrin signaling pathway, synaptic long term depression signaling pathway, opioid signaling pathway, or a combination thereof; and (b) comparing the cf-mRNA levels in the subject to a threshold value of the plurality of cf-mRNAs.

Disclosed herein is a composition for quantifying cell-free messenger RNA (cf-mRNA) levels of a plurality of cf-mRNAs in a biological sample, wherein the plurality of cell-free mRNAs corresponds to a plurality of genes comprising KIAA0100, MAGl1, NNMT, MXD1, ZNF75A, SELL, ASS1, MNDA, and AC132217.4, the composition comprising a plurality of oligonucleotide primers having sequences that hybridize to cDNA sequences transcribed from the plurality of cf-mRNA.

Disclosed herein is a method for detecting a likelihood of a stage of Alzheimer's disease (AD) in a subject, the method comprising: (a) obtaining a biological sample from the subject; and (b) detecting cell-free messenger RNA (cf-mRNA) levels of a plurality of cf-mRNAs in the biological sample, wherein the plurality of cf-mRNAs corresponds to a plurality of genes comprising KIAA0100, MAGI′, NNMT, MXD1, ZNF75A, SELL, ASS1, MNDA and AC132217.4, wherein the method has an accuracy that is greater than 85%. The method can have a sensitivity of at least 80%. The method can have a sensitivity of at least 90%. The method can have a specificity of at least 80%. The biological sample can be blood. The biological sample can be blood serum.

Disclosed herein is a method of assaying an active agent comprising (a) assessing a first cell-free expression profile of a subject at a first time point; (b) administering an active agent to the subject; and (c) assessing a second cell-free expression profile of the subject at a second time point. The method can further comprise comparing the first cell-free expression profile to the second cell-free expression profile. The difference between the first expression profile and the second expression profile can indicate an effect of the therapy. The active agent can be a pharmaceutical compound to treat Alzheimer's disease. The method can further comprise assessing a third cell-free expression profile of a subject at a third time point. Assessing can comprise one or more of sequencing, array hybridization, or nucleic acid amplification. The second time point can be four weeks after the first time point. The method can further comprise assessing a time point every four weeks after the first time point over a period of 18 months. The method can comprise tracking and/or detecting one or more cell-free expression profiles to measure one or more targets of interest for therapy and/or drug discovery and/or development. The method can further comprise measuring pharmacodynamics for a lead optimization and/or a clinical development during therapy and/or drug discovery and development. The method can further comprise creating a profile of gene expression to characterize one or more pharmacodynamic effects associated with an engagement of a specific target for therapy and/or drug discovery and/or development. The method can comprise detecting changes in pharmacodynamics target engagement for therapy and/or drug discovery and development. The subject may have or be suspected of having Alzheimer's disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D show RNA concentrations and gene-expression profile sample distribution. FIG. 1A illustrates a typical Bioanalyzer profile of RNA extracted from plasma (top). RNA concentration of RNA extracted from AD and NCI plasma. FIG. 1B shows a histogram of Pearson's correlation coefficient between two replicates. FIG. 1C shows a principal component analysis of all sequenced samples. FIG. 1D shows a principal component analysis of all sequenced samples after correction.

FIGS. 2A-2D show that the cell-free messenger ribonucleic acid (cf-mRNA) sequencing is a comprehensive and accurate approach for characterizing cf-mRNA transcriptome. FIG. 2A shows a histogram of transcripts detected per sample. FIG. 2B shows a histogram of Pearson's correlation coefficient with spiked-in endogenous control. FIG. 2C shows an example of correlation between replicates for individual transcripts using Pearson's correlation analysis.

FIG. 2D shows an aggregated coverage across all the exon-intron junctions of consistently detected genes (TPM>5 in all NCI controls, 3490 genes in total).

FIGS. 3A-3C show a transcriptional landscape of cf-mRNA in AD patients and functional implications based on gene-set analysis and functional annotations. FIG. 3A shows a schematic of the study design. FIG. 3B shows a volcano plot of differentially expressed genes in cf-mRNA between AD (n=126) and NCI controls (n=115). FDR<0.05 was used as the cut-off criteria. FIG. 3C shows the most significant pathways identified using gene set enrichment analysis (top, upregulated genes; bottom, downregulated genes). The black vertical dotted line represents significance threshold (p<0.05).

FIGS. 4A-4C show biological processes and signaling pathways that are associated with AD. FIG. 4A shows biological processes determined by IPA analysis for genes that are upregulated in cf-mRNA of AD as input (left). Most prominent biological processes determined by IPA analysis for genes that are downregulated in cf-mRNA of AD as input (right). FIG. 4B shows subcategories within nervous system development and function (IPA) for genes that are downregulated in cf-mRNA of AD as input. FIG. 4C shows biological processes determined by Gene Ontology for genes that are upregulated in cf-mRNA of AD as input (left) and the most prominent biological processes determined by Gene Ontology for genes that are downregulated in cf-mRNA of AD as input (right).

FIGS. 5A-5C show cf-mRNA transcripts significantly overlap with brain tissue transcripts and transcripts that are dysregulated in AD. FIG. 5A shows overlap between the Genotype-Tissue Expression (GTEx) defined brain enriched genes and downregulated genes in cf-mRNA of AD (left) and overlap between GTEx defined liver enriched genes and downregulated genes in cf-mRNA of AD (right). P-values show comparison between number of overlapped genes versus expected number. FIG. 5B shows overlap between genes that are upregulated in cf-mRNA of AD compared to NCI against genes that are upregulated in the brain tissue of AD patients (left). FIG. 5C shows overlap between genes that are downregulated in cf-mRNA of AD compared to NCI against genes that are downregulated in the brain tissue of AD patients (left).

FIGS. 6A-6E illustrate that cf-mRNA classifier robustly distinguishes AD from NCI. FIG. 6A shows a schematic of classifier establishment. FIG. 6B shows an evaluation of classification accuracy using training cohort. The y-axis depicts AUROC of individual algorithms. FIG. 6C shows a ROC curve of cf-mRNA classifier for discriminating AD against NCI (left) and a waterfall plot of AD and NCI discrimination (right). FIG. 6D shows a ROC curve of a 9-gene mini classifier for discriminating AD against NCI. FIG. 6E shows read counts between AD and NCI in total cohort (123 AD and 114 NCI) for 9 mini-classifier genes.

FIG. 7A illustrates the expression levels of 1,496 dysregulated genes in AD patients with CDR≤1 (FDR<0.05). FIG. 7B shows genes downregulated in “early stage” AD patients are primarily enriched in nervous system function and developmental processes (e.g., Netrin signaling, CREB signaling in neurons, Calcium transport, and Regulation of neurogenesis) and upregulated genes in immune response and proteostasis (e.g., protein ubiquitination, inflammasome pathway, and activation of immune response).

FIGS. 8A-8G show that cf-mRNA genes correlate with severity of cognitive impairment. FIG. 8A shows that a consensus matrix NMF clustering identifies size biologically distinct clusters. Unsupervised NMF clustering from 2591 differentially expressed genes. FIG. 8B shows the expression of “synaptic transmission” and “immune & inflammatory response” clusters categorized by CDR rating. FIG. 8C shows a plot between FDR (represented as −log) and Pearson's correlation coefficient for CDR and TPM of genes. Red dotted line represents FDR=0.05. FIG. 8D shows top canonical pathways identified in IPA pathway analysis using 706 genes that correlate with CDR scores. Red dotted line represents FDR=0.05. FIG. 8E shows the expression of SLU7 based on CDR and MMSE scores (CDR scores (top) and MMSE (bottom)). FIG. 8F shows an average ROC curve of the cf-mRNA classifier for distinguishing NCI (CDR=0) against those with CDR score of 0.5-1. 15 iterations of cross-validation were performed, and the curve represents the average of those 15 ROC curves. FIG. 8G shows unsupervised clustering of AD patients using their cf-mRNA profile based on NMF clusters identified in FIG. 8A.

FIGS. 9A-9C show the expression of cf-mRNA genes against cognitive impairment scores. FIG. 9A illustrates cluster values for each of the 5 AD patient subcategories, Age and MMSE distribution among 5 patient groups identified using ANOVA analysis-Tukey's post-hoc test. FIG. 9B shows a plot between FDR (represented as −log) and Pearson's correlation coefficient for MMSE vs TPM of genes. Red dotted line represents FDR=0.05. FIG. 9C shows top canonical pathways identified in IPA pathway analysis using 520 genes that correlate with MMSE scores. Red dotted line represents FDR=0.05. FIG. 9D shows overlapping genes between genes that correlate with MMSE and CDR scores.

FIG. 10 depicts a computer system consistent with the disclosure herein.

FIG. 11 shows the differential expression of TCF7 in Transcripts per Million (TPM) by age group.

FIG. 12 shows the differential expression of PTK2 (focal adhesion kinase in senescent cells) in TPM by age group.

FIG. 13 shows the differential expression of FER in TPM by age group.

FIG. 14 shows the differential expression of CD36 in TPM by age group. CD36 is one of 18 genes of the panel G00000302 “response to reactive oxygen species” function which correlates with age.

FIG. 15 shows the differential expression of WWTR1 in TPM by age group. WWTR1 is expressed in the Hippo pathway in connection with the YAP/TAZ complex. WWTR1 is one of 40 non-blood genes which correlate with age.

FIG. 16 shows the differential expression of CAV1 in TPM by age group. CAV1 is Caveolin 1 involved in caveolae formation. CAV1 is one of 40 non-blood genes which correlate with age.

FIG. 17 shows a comparison of age-associated genes with other data sets. Two genes, NELL2 and LTB, are consistently highly correlated with age.

FIG. 18 shows a heat map of the expression of 41 age associated genes which overlap with non-blood genes with a p-value of 3.93e-11.

FIG. 19 shows a chart of age associated genes for multiple tissues using GTEx data.

### DETAILED DESCRIPTION

Methods, systems, and kits described herein relate to the rapid, noninvasive detection of disorders using a combination of marker types so as to concurrently determine both a likely disorder and a likely tissue under duress, taking into account changes in gene expression brought about by the natural aging of an individual. In some embodiments, a gene panel comprising genes known to be differentially expressed in individuals at the age of a subject is applied to a cell-free RNA (cfRNA) expression profile of the subject. Through practice of the disclosure herein, one can make predictions as to a disease identity, and the extent of its impact on one or more tissues, without invasive investigation of the tissue or tissues suspected of being impacted.

There is a need to develop a reliable and non-invasive test to accurately diagnose Alzheimer's disease earlier on. Physicians often use a numeric scale, Clinical Dementia Rating (CDR), to quantify the severity of a neurodegenerative disorder. Further, the Mini-Mental State Exam (MMSE) or the Folstein test is used in clinical and research settings to measure cognitive impairment.

The identification of disease markers in circulation, such as in a blood sample, can be a useful tool allowing for the identification of diseased tissue without the need for invasive procedures such as a biopsy. This can be useful in older populations who may be less resilient to such invasive, painful procedures. Factors other than disease which may affect gene expression can also be taken into account. The gene expression of some tissues changes as individual ages. It may be important to identify gene markers associated with age and how they are differentially expressed in order to take them into account when diagnosing a diseased tissue

Here, by performing a transcriptome-wide comparison of plasma cf-mRNA profiles between age matched AD patients and control individuals, proof-of-concept is shown that the circulating transcriptome has the potential to reveal, in a non-invasive manner, molecular and functional information of neurodegenerative diseases such as AD. Technical performance of the assay is disclosed herein, as well as detection and quantification of thousands of genes in circulation to show that genes dysregulated in the plasma of AD patients can reflect biological processes and pathways known to be associated with cognitive impairment and neurodegenerative disorders. For example, disclosed herein is an overall decline in AD patients of multiple pathways implicated in the nervous system function and development (e.g., synapse loss, GABA signaling, and neurotransmission), accompanied by elevated levels of genes involved in inflammation, mitochondrial dysfunction, oxidation, and proteostasis. Further, the genes and biological processes found to be dysregulated in the plasma of AD patients substantially overlapped with those identified in the RNA-seq datasets from postmortem brain biopsy specimens. Cell free-mRNA in plasma can be a surrogate for non-invasive molecular evaluation of brain homeostasis in AD patients.

One potential application that would benefit from a better understanding of the molecular mechanisms involved in AD, is the development of new therapeutic strategies. cf-mRNA sequencing can provide a granular characterization of AD patients' circulating transcriptome, including thousands of genes that are either dysregulated in AD patients or correlated with AD severity. In addition to showing high resolution on biological processes already known to be linked to AD (e.g., 26 dysregulated genes involved in GABA signaling), reduced levels of genes associated with neurogenesis in AD patients were observed, which, without being bound by any one particular theory, may support the hypothesis of adult neurogenesis being disrupted in AD. Further, many factors involved in RNA splicing were identified to be dysregulated in AD patients, such as SLU7, whose levels strongly correlate with disease severity. Evidence points to a role of alternative RNA splicing in aging and neurodegeneration. A prominent decrease of netrin signaling in AD patients, including a significant reduction in the levels of NETRIN-1, which binds APP and has been proposed as a master regulator of Aβ levels was observed. Decreased NETRIN-1 expression is associated with increased Aβ concentration. The integrated cf-mRNA technology solution can provide an approach to better understand the heterogeneous etiology of AD and may aid in the identification of new molecular entities with therapeutic potential and increase their probability of technical success in pre-clinical and clinical stages.

Indeed, the heterogeneous nature of AD, as a complex neurodegenerative disease affecting multiple biological pathways and processes during its onset and progression, represents one major difficulty for AD drug development. So far, therapeutic drugs targeting β-amyloids and tau proteins have shown modest results, therefore multiple compounds targeting commonly affected pathways in AD, such as inflammation, mitochondrial dysfunction, and neuroprotective compounds are currently being developed and tested as alternatives for AD treatment. Successful development of therapeutic agents for a heterogeneous AD population may rely on the ability to appropriately enrich the trial groups for AD patients likely to respond to the candidate drugs. Since molecular characterization of patients based on brain biopsy is generally not feasible, non-invasive tools that enable pre-selection of patients best suited for each therapy can be useful for clinical trials. The present disclosure indicates that the molecular information revealed by the circulating transcriptome may pave the way to personalized characterization of disease-related processes, thus enabling more efficient patient management and improving the probabilities of success of the interventions. Further, given that cf-mRNA can enable “real time” monitoring of organ health and organ system response to therapeutic interventions, and the repertoire of AD-related processes identified in circulation, an integration of cf-mRNA sequencing and clinical information may also allow monitoring therapy response in AD patients.

Despite post-mortem histology remaining the gold standard for establishing AD pathology, currently CSF, PET, and MRI can be used to diagnose AD patients. However, imaging modalities can be costly and CSF collection can be invasive. Therefore, scalable, accessible, and cost-efficient blood-based tests are desired for the management of AD patients. To date, several protein-based blood biomarkers, including those that measure circulating levels of Aβ peptides, appear to be promising candidates as diagnostic biomarkers for AD, though not without limitations considering that Aβ is also present in individuals without dementia and its levels inconsistently predict the rate of cognitive decline. Profiling the cf-mRNA transcriptome represents a non-invasive approach for the development of molecular classifiers to identify AD patients, as shown by the performance of cf-mRNA based classifiers to discriminate control individuals from AD patients. Therefore, cf-mRNA profiling may offer a novel approach for more personalized patient management that integrates clinical information of disease state with insights on patient-specific molecular characteristics to create solutions for improved patient management. cf-mRNA profiling may aid in clinical trials, for instance, as a potential tool for the discrimination of patients with or without AD, reducing the number of patients who require AP-PET for AD diagnosis, and for stratification of patients with increased likelihood to respond to the therapy based on their molecular characteristics.

Provided herein are noninvasive methods, systems, compositions, and kits for assessing or detecting Alzheimer's disease (AD) in a subject, for example, using a biological sample of the subject. The methods comprise isolating cell-free messenger RNAs (cf-mRNAs) from the biological sample. In some embodiments, the biological sample is a plasma or serum. In other embodiments, the biological sample is cerebrospinal fluid (CSF).

A first transcriptome-wide comparison of plasma cf-mRNA profile between AD and NCI is disclosed herein and cf-mRNA signatures that are distinct to AD are identified. Gene-set enrichment analysis showed that cf-mRNA profile of AD reflected signaling pathways and biological processes that are commonly dysregulated in AD. Furthermore, “immune & inflammatory response” and “synaptic transmission” gene-clusters which correlated with the severity of cognitive impairment are disclosed herein. In addition, genes that are associated with neuronal function, another attribute of AD, are attenuated in cf-mRNA transcriptome of AD patients. Disclosed herein is a set of genes correlated with CDR and MMSE cognitive impairment scores, some of which had substantial gene-expression alteration even in the AD patients with very mild to mild cognitive impairment compared to those that are not cognitively impaired. A classifier which can differentiate AD patients with modest cognitive impairment from normal controls without cognitive impairment, indicating that transcriptional changes in the circulation may be suitable as an early diagnostic tool for AD, is also disclosed herein.

The methods can also employ upfront centrifugation to reduce contamination of unwanted “blood” transcripts from cf-mRNA sequencing data. The methods herein can reduce background noise within the “blood component” blood cells from the tissue-specific cf-mRNA signal. Such noise can increase sequencing depth requirements and dilute signal from tissue-specific cf-mRNA. With this purification step, the cf-mRNA transcripts can be said to be more than likely deriving from a subject's brain. By reducing the background noise with the “blood component” transcripts, the detected cf-mRNA transcripts are likely originated from brain.

Often, serum, plasma, or other biological samples are collected from subjects and the samples are optimized by removing cellular debris. In some embodiments, the samples are collected from subjects at a remote location and are shipped to a testing cite via delivery services. Some subjects are healthy, some experience cognitive impairment, and some are diagnosed with AD. In certain instances, the samples may be enriched in non-blood transcripts. cf-mRNAs including a mixture of genetic materials from different genomic sources, such as cerebrum, cerebellum, dorsal root ganglion, superior cervical ganglion, pineal gland, amygdala, trigeminal ganglion, cerebral cortex, and hypothalamus can be isolated from the optimized samples.

A broad range of centrifugation ranges can be used to optimize the samples so that blood transcripts are removed. In certain cases, the ranges may include 1,500 g to 20,000 g, 1,900 g to 16,000 g, 4,000 g to 16,000 g, 8,000 g to 16,000 g, 10,000 g to 14,000 g, 11,000 g to 13,000 g, 11,500 g to 12,500 g, or suitable lower or higher ranges. In some cases, the sample may be centrifuges at about 12,000 g, essentially 12,000 g, substantially 12,000 g, or 12,000 g. Some ranges span about 12,000 g. Some ranges are within 100 g of 12,000 g. Some centrifugation protocols do not differ substantially from 12,000 g, such as centrifugations at 12,000 g. Alternate ranges having a starting point at a low figure listed above or ending at a high figure listed above are also contemplated. Such centrifugation protocols can contribute to 2.5× improvement in diversity of an RNA library for processing. In various cases, the centrifugation protocols may contribute to a 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9×, 4.0×, or greater than 4.0× improvement in diversity of an RNA library for processing.

Further, cDNAs can be converted based on the isolated cf-mRNAs in order to form a library of cDNAs including a NGS library. For example, cDNAs can be generated from reverse transcription of a cf-mRNA sample. Further, cDNAs can be enriched for quantification.

After building the library of cDNAs, many methods can be used to quantify the levels of different cDNAs. For example, polynucleotide amplification, sequencing, probe hybridization, RT-PCR, and microarray hybridization, among other suitable methods, can be used to quantify levels of cDNAs. Various methods can be used to enrich the cDNAs. For example, some of these methods are based on hybridization to oligonucleotides designed to hybridize to different cDNAs. The hybridization may be to oligonucleotides immobilized on high or low density microarrays, or solution phase hybridization to oligonucleotides modified with a ligand which can be subsequently employed for immobilization of the hybrids to a solid surface, such as a bead. Other methods may employ sequence specific amplification (e.g., PCR) to amplify specific cDNAs in a droplet, allowing amplification of specific cDNAs for downstream sequencing. The droplet-based amplification may enable highly multiplexed PCR without the potential non-specific interaction of a large number of PCR primer pairs and the subsequent generation of non-specific amplification products and reduced amplification efficiency of the cDNAs.

Moreover, differential gene expression can also be identified, or confirmed, using the microarray technique. In this method, polynucleotide sequences of interest (including cDNAs and oligonucleotides) can be plated, or arrayed, on a microchip substrate. The arrayed sequences can be then hybridized with specific DNA probes from cells or tissues of interest.

Further, differential gene expression can also be identified, or confirmed, using the sequencing technique. The polynucleotide sequences of interest (including cDNAs and oligonucleotides) can be used as templates to synthesize sequencing libraries. The libraries can be sequenced, and the reads mapped to an appropriate reference. Exemplary sequencing techniques can include, for example, emulsion PCR, pyrosequencing from Roche 454, semiconductor sequencing from Ion Torrent, SOLiD sequencing by ligation from Life Technologies, sequencing by synthesis from Intelligent Biosystems, bridge amplification on a flow cell (e.g., Solexa/Illumina), isothermal amplification by Wildfire technology (Life Technologies), or rolonies/nanoballs generated by rolling circle amplification (Complete Genomics, Intelligent Biosystems, Polonator). Sequencing technologies such as Heliscope (Helicos), SMRT technology (Pacific Biosciences), or nanopore sequencing (Oxford Nanopore), which can allow direct sequencing of single molecules without prior clonal amplification, may be suitable sequencing platforms. Other sequencing methods are also within the scope of this disclosure. Sequencing may be performed with or without target enrichment. Moreover, RT-PCR can be used to quantify different gene expression levels. Generally, the reverse transcription reaction step can be primed using specific primers, random hexamers, or oligo-dT primers, depending on the goal of expression profiling. Reverse transcriptases can be avian myeloblastosis virus reverse transcriptase (AMV-RT), Moloney murine leukemia virus reverse transcriptase (MLV-RT), or other suitable reverse transcriptases.

Although the PCR step can use a variety of thermostable DNA-dependent DNA polymerases, it typically employs the Taq DNA polymerase, which can have a 5′-3′ nuclease activity but lacks a 3′-5′ proofreading endonuclease activity. Thus, TaqMan™ PCR typically utilizes the 5′-nuclease activity of Taq or Tth polymerase to hydrolyze a hybridization probe bound to its target amplicon, but any suitable enzyme with equivalent 5′ nuclease activity can be used. Two oligonucleotide primers can be used to generate an amplicon typical of a PCR reaction. A third oligonucleotide, or probe, can be designed to detect nucleotide sequence located between the two PCR primers. The probe can be non-extendible by Taq DNA polymerase enzyme, and can be labeled with a reporter fluorescent dye and a quencher fluorescent dye. Any laser-induced emission from the reporter dye can be quenched by the quenching dye when the two dyes are located close together, for example, as they are on the probe. During the amplification reaction, the Taq DNA polymerase enzyme can cleave the probe in a template-dependent manner. The resultant probe fragments can disassociate in solution, and signal from the released reporter dye can be freed from the quenching effect of the second fluorophore. One molecule of reporter dye can be liberated for each new molecule synthesized, and detection of the unquenched reporter dye can provide basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif, USA) or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system comprises a thermocycler, laser, charge-coupled device (CCD), camera, and computer. The system includes software for running the instrument and for analyzing the data. 5′-nuclease assay data can initially be expressed as Ct (the threshold cycle). Fluorescence values can be recorded during every cycle and represent the amount of product amplified to that point in the amplification reaction. The point when the fluorescent signal is first recorded as statistically significant can be the threshold cycle (Ct).

### Panel of Differentially Expressed Genes

The biomarker panels comprising a plurality of differentially expressed protein encoding genes described herein can facilitate a sensitive and non-intrusive testing to detect whether a subject has AD or to determine the clinical development stage of AD. Clinical development stages of Alzheimer's disease include (1) preclinical Alzheimer's disease, (2) mild cognitive impairment due to Alzheimer's disease, (3) mild dementia due to Alzheimer's disease, (4) moderate dementia due to Alzheimer's disease, and (5) severe dementia due to Alzheimer's disease. Biomarker panels comprising a plurality of differentially expressed protein encoding genes are often readily obtained by a blood draw from an individual. Benefits of using the biomarker panels disclosed herein can include fast and convenient detecting of AD without cumbersome and unreliable testing.

Biomarker panels as disclosed herein can be selected such that their predictive value as panels is substantially greater than the predictive value of their individual members. Panel members generally do not co-vary with one another, such that panel members provide independent contributions to the panel's overall health signal. Biomarker panels can comprise genes dysregulated in plasma of AD patients, as well as genes that correlated with disease severity, that are enriched in biological processes associated with AD, such as synaptic dysfunction, mitochondrial dysfunction, and inflammation. Genes dysregulated in circulation can be used to identify AD patient subtypes among a heterogeneous population patients, and build cf-mRNA based classifiers that discriminate (e.g., robustly discriminate) age matched controls from AD patients. Cell-free mRNA biomarker panels can non-invasively reveal molecular characteristics associated with neurodegeneration and AD, and support the potential of integrating cf-mRNA with clinical information to potentially improve the AD patient management, identify new therapeutic targets, and enable patient stratification to increase the probability of technical success of the research and development of therapeutics. Accordingly, a panel may be able to substantially outperform the performance of any individual constituent indicative of an individual's AD status, such that a commercially and medicinally relevant degree of confidence (such as sensitivity, specificity, or sensitivity and specificity) is obtained.

In some cases, panel members vary independently from each other. As a result, panels herein often indicate a health risk despite the fact that one or more than one individual members of the panel would not indicate that the health risk is present if measured alone. In other cases, panels herein indicate a health risk at a significant level of confidence despite the fact that no individual panel member indicates the health risk at a significant level of confidence on its own. In yet other cases, panels herein can indicate a health risk at a significant level of confidence despite the fact that at least one individual member indicates at a significant level of confidence that the health risk is not present.

Some biomarker panels comprise some or all of the differentially expressed protein encoding genes recited herein (see Table 1A). In some cases, a biomarker panel may comprise at least nine protein encoding genes. In some cases, the biomarker panel may comprise any two genes from Table 1A. In some cases, the biomarker panel may comprise any three genes from Table 1A. In some cases, the biomarker panel may comprise any four genes from Table 1A. In some cases, the biomarker panel may comprise any five genes from Table 1A. In some cases, the biomarker panel may comprise any six genes from Table 1A. In some cases, the biomarker panel may comprise any seven genes from Table 1A. In some cases, the biomarker panel may comprise any eight genes from Table 1A. In some cases, the biomarker panel may comprise the nine genes from Table 1A.

TABLE 1A
List of differentially expressed genes

| Ref. No. | Gene names |
|---|---|
| 1 | KIAA0100 |
| 2 | MAGl1 |
| 3 | NNMT |
| 4 | MXD1 |
| 5 | ZNF75A |
| 6 | SELL |
| 7 | ASS1 |
| 8 | MNDA |
| 9 | AC132217.4 |

In addition, some biomarker panels may comprise some or all of the differentially expressed protein encoding genes recited herein (see Table 1B). In some cases, a biomarker panel may comprise at least 14 protein encoding genes. In some cases, the biomarker panel may comprise any two genes from Table 1B. In some cases, the biomarker panel may comprise any three genes from Table 1B. In some cases, the biomarker panel may comprise any four genes from Table 1B. In some cases, the biomarker panel may comprise any five genes from Table 1B. In some cases, the biomarker panel may comprise any six genes from Table 1B. In some cases, the biomarker panel may comprise any seven genes from Table 1B. In some cases, the biomarker panel may comprise any eight genes from Table 1B. In some cases, the biomarker panel may comprise any nine genes from Table 1B. In some cases, the biomarker panel may comprise any ten genes from Table 1B. In some cases, the biomarker panel may comprise any eleven genes from Table 1B. In some cases, the biomarker panel may comprise any twelve genes from Table 1B. In some cases, the biomarker panel may comprise any thirteen genes from Table 1B. In some cases, the biomarker panel may comprise the fourteen genes from Table 1B.

TABLE 1B
List of additional differentially expressed genes

| Ref. No. | Gene names |
|---|---|
| 1 | SLU7 |
| 2 | HNRNPA2B1 |
| 3 | GGCT |
| 4 | NDUFA12 |
| 5 | HSPB11 |
| 6 | ATP6V1B2 |
| 7 | SASS6 |
| 8 | SUMO1 |
| 9 | KRCC1 |
| 10 | LSM6 |
| 11 | LCP1 |
| 12 | SASS6 |
| 13 | ATP6v1B2 |
| 14 | MAT2B |

After construction of various biomarker panels, the biomarker panels can be used to determine whether a subject has AD as described in the non-invasive diagnostic methods provided herein. Further, the biomarker panels can also be used to determine a particular development stage of AD. Often, different development stages of AD are assigned with either a CDR score or a MMSE score. Some of the methods herein comprise comparing a level of a biomarker panel in a subject to a threshold level of the same biomarker panel. In some cases, the threshold level of a biomarker panel equals the level of the biomarker panel of a control subject. In some cases, the control subject is a person having a known diagnosis. For example, the control subject can be a negative control subject. The negative control subject can be a subject that does not have AD. For other example, the control subject can be a positive control subject. The positive control subject can be a subject having a confirmed diagnosis of AD. The positive control subject can be a subject having a confirmed diagnosis of AD. Further, the positive control subject can be a subject having a confirmed diagnosis of any stage of AD. For example, the positive control subject may have a CDR score of 0.5, 1, 2, or 3. The positive control subject may have a MMSE score of 1-6, 6-12, 12-18, 18-24, or 24-30. The threshold value can be a predetermined level of the biomarker, wherein the predetermined level is set based upon a measured amount of the biomarker in a control subject.

Diagnostic methods described herein for detection of AD in a subject can detect AD with a sensitivity greater than 75%, greater than 80%, greater than 85%, greater than 90%, greater than 95%, greater than 96%, greater than 97%, greater than 98%, greater than 99%, or about 100%. Such diagnostic methods can detect Alzheimer's Disease (AD) with a sensitivity that is 70% to 100%, 80% to 100%, or 90% to 100%. Such diagnostic methods can detect AD with a specificity greater than 70%, greater than 75%, greater than 80%, greater than 85%, greater than 90%, greater than 95%, greater than 96%, greater than 97%, greater than 98%, greater than 99%, or about 100%. Such diagnostic methods can detect AD with a specificity that is from 50% to 100%, from 60% to 100%, from 70% to 100%, from 80% to 100%, or from 90% to 100%. In various embodiments, such diagnostic methods can detect AD with a sensitivity and a specificity that is 50% or greater, 60% or greater, 70% or greater, 75% or greater, 80% or greater, 85% or greater, or 90% or greater. In certain embodiments, such diagnostic methods can detect AD with a sensitivity and a specificity that is 50% to 100%, 60% to 100%, 70% to 100%, 80% to 100%, or 90% to 100%.

### Classifier

Classifiers can be developed using many different technologies. For example, computer systems can be used to develop and generate classifiers. Data, such as cf-mRNA levels, collected from the plurality of differentially expressed protein coding genes can be used to train a machine learning algorithm to obtain a classifier.

Machine learning can be generalized as the ability of a learning machine to perform accurately on new, unseen examples/tasks after having experienced a learning data set. Machine learning may include the concepts and methods provided herein. Supervised learning concepts may include: AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines (SVM); Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; logistic regression with L1 regularization (LASSO); logistic regression with L2 regularization (ridge classifier); Decision trees, such as C4.5, Random forests, ID3, CART, SLIQ, SPRINT; Bayesian networks, such as Naive Bayes; and Hidden Markov models. Unsupervised learning concepts may include: Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as K-means algorithm, Fuzzy clustering, DBSCAN, and OPTICS algorithm; and Outlier Detection, such as Local Outlier Factor. Semi-supervised learning concepts may include: Generative models, Low-density separation, Graphbased methods, and Co-training. Reinforcement learning concepts may include: Temporal difference learning, Q-learning, Learning Automata, and SARSA. Deep learning concepts may include: Deep belief networks, Deep Boltzmann machines, Deep Convolutional neural networks, Deep Recurrent neural networks, and Hierarchical temporal memory.

In some cases, the performance of a classifier is assessed in some cases via the AUC of the ROC as reported herein. A ROC considers the performance of the classifier at all possible model score cutoff points. However, when a classification decision needs to be made (e.g., is this patient sick or healthy?), a cutoff point is used to define the two groups. Classification scores at or above the cutoff point are assessed as positive (or sick) while points below are assessed as negative (or healthy) in various embodiments.

For some classification models disclosed herein, a classification score cutoff point is established by selecting the point of maximum accuracy on the validation ROC. The point of maximum accuracy on an ROC is the cutoff point or points for which the total number of correct classification calls is maximized. Here, the positive and negative classification calls are weighted equally. In cases where multiple maximum accuracy points are present on a given ROC, the point with the associated maximum sensitivity may be selected.

### Clinical Outcome Score

Machine learning algorithms for sub-selecting discriminating biomarkers and/or subject characteristics, and for building classification models, are used in some methods and systems herein to determine clinical outcome scores. These algorithms include, but are not limited to, elastic networks, random forests, support vector machines, and logistic regression. These algorithms can aid in selection of important biomarker features and transform the underlying measurements into a score or probability relating to, for example, clinical outcome, disease risk, disease likelihood, presence or absence of disease, treatment response, and/or classification of disease status.

A clinical outcome score can be generated by inputting quantified cf-mRNA levels to a classifier described herein. Also, a clinical outcome score is determined by comparing cf-mRNA levels that corresponds to at least two differentially expressed genes in the biological sample obtained from the subject to a reference cf-mRNA level of the two genes. Alternately or in combination, a clinical outcome score is determined by comparing a subject-specific profile of a panel of cf-mRNA levels correspond to differentially expressed genes to a reference profile of the differentially expressed genes. Often, a reference level or reference profile represents a known diagnosis. For example, a reference level or reference profile represents a positive diagnosis of AD. As another example, a reference level or reference profile represents a negative diagnosis of AD. Similarly, a reference level or reference profile represents a particular score associated with CDR or MMSE.

In some cases, an increase in a score indicates an increased likelihood of one or more of a: poor clinical outcome, good clinical outcome, high risk of disease, low risk of disease, complete response, partial response, stable disease, non-response, and recommended treatment (or treatments) for disease management. In some cases, a decrease in the quantitative score indicates an increased likelihood of one or more of a: poor clinical outcome, good clinical outcome, high risk of disease, low risk of disease, complete response, partial response, stable disease, non-response, and recommended treatment (or treatments) for disease management. Also, in some embodiments, an increase in a score indicates a higher CDR or MMSE score.

A similar profile from a patient to a reference profile often indicates an increased likelihood of one or more of a: poor clinical outcome, good clinical outcome, high risk of disease, low risk of disease, complete response, partial response, stable disease, non-response, and recommended treatment (or treatments) for disease management. In some applications, a dissimilar biomarker profile from a patient to a reference profile may indicate one or more of: an increased likelihood of a poor clinical outcome, a good clinical outcome, a high risk of disease, a low risk of disease, a complete response, a partial response, a stable disease, a non-response, and a recommended treatment (or treatments) for disease management.

An increase threshold values of cf-mRNA levels corresponding to one or more differentially expressed genes often indicates an increased likelihood of one or more of a: poor clinical outcome, good clinical outcome, high risk of disease, low risk of disease, complete response, partial response, stable disease, non-response, and recommended treatment (or treatments) for disease management. In some applications, a decrease in one or more biomarker threshold values may indicate an increased likelihood of one or more of a: poor clinical outcome, good clinical outcome, high risk of disease, low risk of disease, complete response, partial response, stable disease, non-response, and recommended treatment (or treatments) for disease management.

An increase in at least one of a quantitative score, one or more thresholds, or similar biomarker profile values indicates an increased likelihood of one or more of a: poor clinical outcome, good clinical outcome, high risk of disease, low risk of disease, complete response, partial response, stable disease, non-response, and recommended treatment (or treatments) for disease management. Similarly, a decrease in at least one of a quantitative score, one or more biomarker thresholds, similar biomarker profile values or combinations thereof indicates an increased likelihood of one or more of a: poor clinical outcome, good clinical outcome, high risk of disease, low risk of disease, complete response, partial response, stable disease, non-response, and recommended treatment (or treatments) for disease management.

### Treatment and Monitoring Regimens

Provided herein are diagnostic, monitoring, and treatment regimens for implementing any of the methods described herein for detecting a presence or absence of AD and/or treatment of the same.

For example, Mini-Mental State Exam (MMSE) can be administered to assess whether there are problems with areas of a subject's brain involved in learning, memory, thinking, or planning skills. Alternatively or additionally, computed tomography (CT) scan can be used to monitor brain changes that are common in the later stages of Alzheimer's. Similarly, magnetic resonance imaging (MRI), CSF, and PET can be helpful to measure amyloid markers to monitor the brain changes that are linked to AD. Alternatively or additionally, neuropsychological testing can be administered to monitor the relationship between the brain and behavior. Neuropsychological testing can help diagnosis of conditions that affect thinking, emotion, and behavior, including AD.

A number of treatment methods are contemplated here as well. Different types of drugs can treat memory loss, behavior changes, sleep problems, and other AD's symptoms. For example, citalopram, fluoxetine, paroxetine, and sertraline can be used to treat problems with mood, depression, and irritability experienced by AD patients. Alprazolam, buspirone, iorazepam, and oxazepam can be used to treat anxiety or restlessness associated with AD. Alternatively or additionally, cholinesterase inhibitors and/or memantine can be administered to alleviate symptoms associated with AD. Further, unconventional therapies, such as hormone replacement therapy, art and music therapies, and supplements (e.g., vitamin E) can be used alternatively or additionally to treat AD.

Methods, systems, and kits disclosed herein can be intended to non-invasively detect a tissue or organ in a subject that is under duress as well as determine which disease or condition is affecting the tissue or organ under duress. In some instances, the methods, systems and kits can provide for treating a subject for a disease or condition. Some methods disclosed herein can comprise selecting a method or therapy for treating a subject for a disease or condition. Some kits and systems disclosed herein can provide for selecting a method or therapy for treating a subject for a disease or condition. Some methods disclosed herein comprise monitoring a disease or condition in a subject, or administering a test for a disease or condition. Some kits and systems disclosed herein provide for monitoring a disease or condition in a subject, or administering a test for a disease or condition. Some methods disclosed herein comprise treating a subject for a disease or condition, monitoring a disease or condition in a subject, or administering a test for a disease or condition. In some instances, the methods disclosed herein comprise determining the subject has a disease or condition, thereby informing the subject or their healthcare provider that a treatment or test would be appropriate, suitable, or beneficial to the subject. In some instances, the methods disclosed herein comprise determining the subject has a disease or condition and recommending a treatment for the disease or condition. In some instances, the methods disclosed herein comprise determining the subject has a disease or condition and treating the subject for the disease or condition. In some instances, the methods disclosed herein comprise determining the subject has a disease or condition and monitoring the subject for the disease or condition. In some instances, the methods disclosed herein comprise determining the subject has an increased risk or possibility of having the disease or condition relative to an individual within the same age range without the disease or condition, and administering a test specific for the disease or condition to the subject. In some instances, the methods disclosed herein comprise determining the subject has an increased risk or possibility of having the disease or condition relative to an individual within the same age range without the disease or condition, and recommending a test specific for the disease or condition to the subject.

Provided herein are therapeutic agents, compositions, compounds, and agents for the treatments of diseases and conditions. Combinations and analogs of these agents are contemplated and intended herein even if each combination and analog is not explicitly described. An “analog,” as used herein, generally refers to a modified or synthetic compound that resembles a naturally occurring compound, wherein at least 50% of the analog structure is identical to at least 50% of the naturally occurring compound.

Disease presence and location in a subject can be determined at an early stage of disease with greater accuracy, because the systems and methods described herein provide rapid results, take into account gene expression variations by age, and are non-invasive and inexpensive. Thus, the subject can be advantageously treated before the disease progresses to advanced stages that are relatively more difficult to control or treat as compared to early stages. For example, the systems and methods disclosed herein may allow for determining which tissue(s) or organ(s) are showing signs of neurodegeneration before the onset of symptoms. In this way, the methods and systems disclosed herein can provide for focused analysis and targeted therapies at early stages of disease.

The methods and systems can provide for treating a subject with a therapy that is suitable or optimal for the extent of tissue damage. In some instances, the methods may comprise detecting the markers and/or tissue-specific polynucleotides to assess the effectiveness or toxicity of a therapy. In certain instances, the methods may comprise quantifying the markers and/or tissue-specific polynucleotides to assess the effectiveness or toxicity of a therapy. In some instances, the therapy is continued. In various instances, the therapy is discontinued. In certain instances, the therapy is replaced with another therapy. Regardless, due to the rapid and non-invasive nature of the methods and systems, therapeutic effects can be assessed and optimized more often relative to conventional treatment optimization.

In some aspects, the present disclosure provides for uses of the systems, samples, markers, and tissue-specific polynucleotides disclosed herein. In some instances, disclosed herein are uses of an in vitro sample for non-invasively detecting a tissue or organ in a subject that is under duress and as well as a disease or condition that is the cause of the duress. In some instances, disclosed herein are uses of an ex vivo sample for non-invasively detecting a tissue or organ in a subject that is under duress and as well as a disease or condition that is the cause of the duress by comparing the gene expression data to an age-dependent expression control. Generally, uses disclosed herein comprise quantifying markers and tissue-specific polynucleotides in samples, including ex vivo samples and in vitro samples. Some uses disclosed herein comprise comparing a quantity of a marker and a quantity of tissue-specific polynucleotide in a first sample and comparing the quantities to respective quantities in a second sample. In some instances, the first sample is from a first subject and the second sample is from a control subject (e.g., a healthy subject or subject with a condition wherein the subject is in the same age range as the first subject). In some instances, the first sample is from a subject at a first time point and the second sample is from the same subject at a second time point. The first time point may be obtained before the subject is administered a therapy and the second time point may be obtained after the therapy. Thus, also provided herein are uses of samples, markers, tissue-specific polynucleotides, kits, and systems disclosed herein to monitor or evaluate a condition of a subject, tissue health state of a subject, or an effect of a therapeutic agent.

In some aspects, the disclosure provides for methods of monitoring a human subject with a chronic condition for a presence of at least one complication of at least one tissue. In some aspects, the disclosure provide for methods of monitoring a human subject with a chronic condition for an increased risk of at least one complication of at least one tissue.

Some methods comprise monitoring the human subject for a complication in any one of at least three tissues. Some methods comprise monitoring the human subject for an increased risk of a complication in any one of at least three tissues.

Gene expression panels as disclosed herein can share a property that sensitive, specific conclusions regarding an individual's tissue disease state are made using cfRNA expression level information derived from circulating blood in combination with knowledge of the individual's age. A benefit of the present gene marker panels is that they provide a sensitive, specific tissue health assessment using conveniently, noninvasively obtained samples. There may be no need to rely upon additional data obtained from intrusive biopsies. As a result, compliance rates may be substantially higher and tissue health issues are more easily recognized early in their progression, so that they may be more efficiently treated.

### Cell Type and Tissue Type Specific Polynucleotides

Provided herein are kits, devices, systems, and methods employing cell type-specific gene expression, cell type-specific nucleic acids (e.g., RNAs) and cell type-specific nucleic acid modifications (e.g., methylation patterns) disclosed herein. The terms, “cell type-specific nucleic acid,” “cell type-specific polynucleotide,” “tissue-specific nucleic acid,” and “tissue-specific polynucleotide” are interchangeable as used herein. The term “cell type-specific” may be used to characterize a nucleic acid that is expressed in a single tissue of the subject. Alternatively, the term “cell type-specific” may be used to characterize a nucleic acid that is predominantly expressed in a specific cellular function or signaling pathway disclosed herein. The cellular function or pathway can include neuroinflammation, immune response, hypoxia signaling, production of nitric oxide, systemic lupus erythematosus signaling, toll-like receptor signaling, NG-kappaB signaling, inflammasome pathway, mitochondrial dysfunction, protein ubiquitination, etc. For the purposes of this application, predominantly expressed may mean that the tissue-specific nucleic acid is expressed at an RNA level that is at least 50% greater in the specific tissue than the RNA level of the tissue-specific nucleic acid in any other tissue of the subject. However, in some cases, a tissue-specific nucleic acid expressed at an RNA level that is at least 30% greater in the specific tissue than that of any other tissue may be sufficient for the methods disclosed herein. In other cases, a tissue-specific nucleic acid expressed at an RNA level that is at least 80% greater in the specific tissue than that of any other tissue may be required by the methods disclosed herein. Predominantly expressed may mean that the tissue-specific nucleic acid is expressed at an RNA level that is at least 2-fold greater in the specific tissue of interest than the RNA level of the tissue-specific nucleic acid in any other tissue of the subject. Predominantly expressed may mean that the tissue-specific nucleic acid is expressed at an RNA level that is at least 5-fold greater in the specific tissue of interest than the RNA level of the tissue-specific nucleic acid in any other tissue of the subject. Predominantly expressed may mean that the tissue-specific nucleic acid is expressed at an RNA level that is at least 10-fold greater in the specific tissue of interest than the RNA level of the tissue-specific nucleic acid in any other tissue of the subject. Predominantly expressed may mean that a detectable amount of the tissue-specific nucleic acid would occur in a biological fluid (e.g., plasma) of the subject only when damage occurs to the specific tissue where the tissue-specific nucleic acid is predominantly expressed.

Provided herein are kits, systems, and methods for detecting or quantifying a biological molecule in a sample from a subject, including by way of non-limiting example, polynucleotides, peptides/proteins, lipids, and sterols. Biological molecules disclosed herein may be tissue-specific. The term “tissue-specific,” as used herein, generally refers to a biological molecule, or modification thereof, that is expressed at a higher level in the single tissue than in any other tissue in the subject. In some instances, it is expressed at least 10% higher in the single tissue than in any other tissue in the subject. In some instances, it is expressed at least 20% higher in the single tissue than in any other tissue in the subject. In some instances, it is expressed at least 30% higher in the single tissue than in any other tissue in the subject. In some instances, it is expressed at least 40% higher in the single tissue than in any other tissue in the subject. In some instances, it is expressed at least 50% higher in the single tissue than in any other tissue in the subject. Thus, the tissue-specific biological molecule may be considered predominantly present or predominantly expressed in a single tissue. Tissue-specific biological molecules disclosed herein may be tissue-specific polynucleotides. Tissue-specific polynucleotides are nucleic acids that are expressed or modified in a tissue-specific manner. For example, there may be only a single tissue or organ, or small set of tissues or organs that predominantly accounts for the expression of a particular gene (e.g., at least 60%, 70%, 80%, 90%, 95%, or more of a gene's total expression in the subject).

Provided herein are kits, systems, and methods for detecting or quantifying a tissue-specific polynucleotide in a sample. At least one database of genetic information can be used to identify a tissue-specific polynucleotide or a panel of tissue-specific polynucleotides. Accordingly, aspects of the disclosure provide systems and methods for the use and development of a database. Methods of the disclosure may utilize databases containing existing data generated across tissue types to identify the tissue-specific genes. Such databases may be utilized for identification of tissue-specific genes. The database may be a web-based gene expression profile. Non-limiting examples of web-based gene expression repositories are publicly available, e.g., The Human Protein Atlas at www_proteinatlas_org, BioGPS at biogps_org and The European Bioinformatics Institute Expression Atlas at www_ebi_ac_uk/gxa/, Gene Expression Omnibus (GEO) at ncbi_nlm_nih_gov/geo/, the content of all of which are incorporated herein by reference. Such databases are also publicly available as published articles in printed and on-line journals. Databases may also include atlases, e.g., the Human 133A/GNF1H Gene Atlas (see Su et al., Proc Natl Acad Sci USA, 2004, vol. 101, pp. 6062-7 for original publication) and RNA-Seq Atlas (see Krupp et al., Bioinformatics, 2012, vol. 15, pp. 1184-5 for original publication), which are both incorporated herein by reference. These databases and websites incorporate data from many independent studies and often corroborate tissue-specific gene expression patterns amongst a species. Such cross-validation can provide useful tissue-specific polynucleotides for methods, systems, and kits disclosed herein. In some instances, a tissue-specific polynucleotide disclosed herein is identified as having tissue-specific expression by at least two published datasets. In some instances, a tissue-specific polynucleotide disclosed herein is identified as having tissue-specific expression by at least three published datasets. In some instances, a tissue-specific polynucleotide disclosed herein is identified as having tissue-specific expression by at least four published datasets. In some instances, a tissue-specific polynucleotide disclosed herein is identified as having tissue-specific expression by at least five published datasets. In order to identify tissue-specific transcripts from at least one database, certain embodiments employ a template-matching algorithm to the databases. Template matching algorithms used to filter data can be used, see, e.g., Pavlidis P, Noble W S (2001) Analysis of strain and regional variation in gene expression in mouse brain. Genome Biol 2:research0042.1-0042.15. Examples of tissue-specific genes include those appearing in FIG. 18 of US20130252835, which is incorporated herein by reference.

Provided herein are kits, systems, and methods for detecting or quantifying a tissue-specific polynucleotide in a sample. The tissue-specific nucleic acid may refer to a nucleic acid that is expressed in a single tissue of each subject in a population of subjects. The tissue-specific nucleic acid may refer to a nucleic acid that is predominantly expressed in a specific tissue of each subject in a population of subjects. The population of subjects may be healthy. The population of subjects may have a common disease or condition. The population of subjects may comprise two subjects. The population of subjects may comprise five subjects. The population of subjects may comprise ten subjects. The population of subjects may comprise twenty subjects. The population of subjects may have a common ethnicity, a common genetic background, a common gender, a common age, or a combination thereof. The tissue-specific nucleic acid may refer to a nucleic acid that is expressed in a single tissue or predominantly expressed in a specific tissue as shown by a published study or database. The published study may have employed microarray technology or RNA-seq profiling to measure tissue-specific nucleic acid levels. In some instances, damage of the specific tissue is caused by a disease or condition resulting in apoptosis of cells in the specific tissue, releasing cell-free tissue-specific nucleic acids into a circulating fluid of the subject. The tissue-specific nucleic acid may be a nucleic acid that is expressed highly enough in the specific tissue that it can be detected in a circulating biological fluid (e.g., blood, plasma) when damage to the specific tissue occurs. The tissue-specific nucleic acid may be a nucleic acid that is expressed highly enough in the specific tissue that it can be detected in a circulating biological fluid (e.g., blood, plasma) when damage to at least 10%, at least 20%, at least 30%, at least 40%, or at least 50% of the specific tissue occurs.

Disclosed herein are methods, kits, and systems for detecting, quantifying, and/or analyzing tissue-specific polynucleotides. In general, the tissue-specific polynucleotides are cell-free polynucleotides, released into a biological fluid (e.g., blood, cerebrospinal fluid, lymphatic fluid, and urine), upon damage or injury to a cell, tissue, or organ. As used herein, damage or injury to the cell, tissue, or organ may be due to a disease or condition that results in disruption of a cell membrane or a loss of cell membrane integrity of the cell or at least one cell within or on the surface of the tissue or organ. Disruption of the cell membrane or loss of cell membrane integrity may result in a release of polynucleotides within the cell. Disruption of the cell membrane may be due, for instance, to necrosis, autolysis, or apoptosis. Non-limiting examples of tissue-specific polynucleotides include tissue-specific RNA, and DNA comprising a tissue-specific methylation pattern. Tissue-specific RNAs may include, but are not limited to, messenger RNA (mRNA), microRNA (miRNA), pre-miRNA, pri-miRNA, pre-mRNA, circular RNA (circRNA), long non-coding RNA (lncRNA), and exosomal RNA. Examples of genes having tissue-specific expression are provided herein.

Provided herein are kits, systems, and methods for detecting or quantifying a biological molecule in a sample from a subject. Biological molecules disclosed herein may be tissue-specific. The term “tissue-specific,” as used herein, generally refers to a biological molecule, or modification thereof, that is expressed at a higher level in the single tissue than in any other tissue in the subject. In some instances, it is expressed at least 10% higher in the single tissue than in any other tissue in the subject. In some instances, it is expressed at least 20% higher in the single tissue than in any other tissue in the subject. In some instances, it is expressed at least 30% higher in the single tissue than in any other tissue in the subject. In some instances, it is expressed at least 40% higher in the single tissue than in any other tissue in the subject. In some instances, it is expressed at least 50% higher in the single tissue than in any other tissue in the subject. Thus, the tissue-specific biological molecule may be considered predominantly present or predominantly expressed in a single tissue. Tissue-specific biological molecules disclosed herein may be tissue-specific polynucleotides. Tissue-specific polynucleotides are nucleic acids that are expressed or modified in a tissue-specific manner. For example, there may be only a single tissue or organ, or small set of tissues or organs that predominantly accounts for the expression of a particular gene (e.g., at least 60%, 70%, 80%, 90%, 95%, or more of a gene's total expression in the subject).

In some instances, methods disclosed herein comprise comparing the level of a single tissue-specific polynucleotide to a corresponding reference level of the tissue-specific polynucleotide is sufficient to determine whether a tissue has been damaged by a disease or condition. In other instances, the level of multiple tissue-specific polynucleotides may be compared to corresponding reference levels of the tissue-specific polynucleotides to determine whether a tissue has been damaged by a disease or condition. The methods disclosed herein may comprise comparing the level of as few as 1, 2, 3, 4, 5, 6, 7, 8, 9, or 10 tissue-specific polynucleotides to corresponding reference levels to determine whether a tissue that has been damaged by a disease or condition. There may be an advantage to comparing as few as 1, 2, or 3 tissue-specific polynucleotides to corresponding reference levels.

In some instances, methods disclosed herein comparing the level of a tissue-specific polynucleotide to a corresponding reference level of the tissue-specific polynucleotide can result in determining that the level of the tissue-specific polynucleotide is greater than the corresponding reference level. In some cases, the corresponding reference level is the level of the tissue-specific polynucleotide in a healthy individual and the level of the tissue-specific polynucleotide being greater than the corresponding reference level is indicative of damage or injury to a specific tissue, organ, or cell in the subject. The level of the tissue-specific polynucleotide may be at least 5%, at least 10%, at least 20%, at least 30%, at least 40%, at least 50%, at least 60%, at least 70%, at least 80%, at least 90%, at least 100%, at least 150%, or at least 200% greater than the corresponding reference level.

In some instances, methods disclosed herein comparing the level of a tissue-specific polynucleotide to a corresponding reference level of the tissue-specific polynucleotide can result in determining that the level of the tissue-specific polynucleotide is lower than the corresponding reference level. In some cases, the corresponding reference level is the level of the tissue-specific polynucleotide in an individual or population having the disease or condition, and the level of the tissue-specific polynucleotide being lower than the corresponding reference level is indicative of the absence or minimal amount of damage or injury to a specific tissue, organ, or cell in the subject. The level of the tissue-specific polynucleotide may be at least 5%, at least 10%, at least 20%, at least 30%, at least 40%, at least 50%, at least 60%, at least 70%, at least 80%, at least 90%, or at least 95% lower than the corresponding reference level.

Tissue-specific polynucleotides disclosed herein may be described as “corresponding to a gene.” In some instances, the phrase “corresponding to a gene” means the tissue-specific polynucleotide is transcribed from a gene. Thus, in some instances, tissue-specific polynucleotides are tissue-specific RNA transcripts. Tissue-specific RNA transcripts include full-length transcripts, transcript fragments, transcript splice variants, enzymatically or chemically cleaved transcripts, transcripts from two or more fused genes, and transcripts from mutated genes. Fragments and cleaved transcripts must retain enough of the full-length polynucleotide to be recognizable as correspond to the gene. In some instances, 5% of the full-length polynucleotide is enough of the full-length polynucleotide. In some instances, 10% of the full-length polynucleotide is enough of the full-length polynucleotide. In some instances, 15% of the full-length polynucleotide is enough of the full-length polynucleotide. In some instances, 20% of the full-length polynucleotide is enough of the full-length polynucleotide. In some instances, 25% of the full-length polynucleotide is enough of the full-length polynucleotide. In some instances, 30% of the full-length polynucleotide is enough of the full-length polynucleotide. In some instances, 40% of the full-length polynucleotide is enough of the full-length polynucleotide. In some instances, 50% of the full-length polynucleotide is enough of the full-length polynucleotide. In some instances, the phrase “corresponding to a gene” means the tissue-specific polynucleotide is a modified form of the gene (e.g., tissue-specific DNA modification pattern).

### Isolating, Quantifying, and Detecting

Often, methods disclosed herein comprise detecting or quantifying an amount of a marker of a disease or condition disclosed herein in to determine that the subject is affected by a respective disease or condition or that the subject is at a risk of being affected by a respective disease or condition. In some instances, detecting or quantifying at least 1 copy/ml of the marker is sufficient to determine that the subject is affected by, or at risk of being affected by, a respective disease or condition. In some instances, detecting or quantifying at least 5 copies/ml of the marker is sufficient to determine that the subject is affected by, or at risk of being affected by, a respective disease or condition. In some instances, detecting or quantifying at least 10 copies/ml of the marker is sufficient to determine that the subject is affected by, or at risk of being affected by, a respective disease or condition. In some instances, detecting or quantifying at least 15 copies/ml of the marker is sufficient to determine that the subject is affected by, or at risk of being affected by, a respective disease or condition. In some instances, detecting or quantifying at least 20 copies/ml of the marker is sufficient to determine that the subject is affected by, or at risk of being affected by, a respective disease or condition. In some instances, detecting or quantifying at least 25 copies/ml of the marker is sufficient to determine that the subject is affected by, or at risk of being affected by, a respective disease or condition. In some instances, detecting or quantifying at least 30 copies/ml of the marker is sufficient to determine that the subject is affected by, or at risk of being affected by, a respective disease or condition. In some instances, detecting or quantifying at least 40 copies/ml of the marker is sufficient to determine that the subject is affected by, or at risk of being affected by, a respective disease or condition. In some instances, detecting or quantifying at least 50 copies/ml of the marker is sufficient to determine that the subject is affected by, or at risk of being affected by, a respective disease or condition. In some instances, detecting or quantifying at least 100 copies/ml of the marker is sufficient to determine that the subject is affected by, or at risk of being affected by, a respective disease or condition.

Often, methods disclosed herein comprise detecting or quantifying an amount of a tissue-specific polynucleotide disclosed herein in to determine that a respective tissue is being affected by a disease or condition. In some instances, methods comprise detecting or quantifying at least 1 copy/ml of the tissue-specific polynucleotide. In some instances, methods comprise detecting or quantifying at least 5 copies/ml of the tissue-specific polynucleotide. In some instances, methods comprise detecting or quantifying at least 10 copies/ml of the tissue-specific polynucleotide. In some instances, methods comprise detecting or quantifying at least 15 copies/ml of the tissue-specific polynucleotide. In some instances, methods comprise detecting or quantifying at least 20 copies/ml of the tissue-specific polynucleotide. In some instances, methods comprise detecting or quantifying at least 25 copies/ml of the tissue-specific polynucleotide. In some instances, methods comprise detecting or quantifying at least 30 copies/ml of the tissue-specific polynucleotide. In some instances, methods comprise detecting or quantifying at least 35 copies/ml of the tissue-specific polynucleotide. In some instances, methods comprise detecting or quantifying at least 40 copies/ml of the tissue-specific polynucleotide. In some instances, methods comprise detecting or quantifying at least 45 copies/ml of the tissue-specific polynucleotide. In some instances, methods comprise detecting or quantifying at least 50 copies/ml of the tissue-specific polynucleotide. In some instances, methods comprise detecting or quantifying at least 100 copies/ml of the tissue-specific polynucleotide.

Some methods disclosed herein comprise detecting or quantifying at least a certain amount of a marker or tissue-specific polynucleotide to determine that a disease or condition is affecting a respective tissue. In some cases, the amount of the marker, wherein the marker is a polynucleotide, or tissue-specific polynucleotide, is at least 1 copy/mL, at least 10 copies/mL, at least 20 copies/mL, at least 30 copies/mL, at least 40 copies/mL, or at least 50 copies/mL, at least 80 copies/cell, at least 100 copies/cell, at least 120 copies/cell, at least 150 copies/cell, or at least 200 copies/cell. In some cases, the amount of the marker, wherein the marker is a protein, lipid, or other non-polynucleotide biological molecule, is at least 5 pg/mL, at least 10 pg/mL, at least 20 pg/mL, at least 30 pg/mL, at least 50 pg/mL, at least 60 pg/mL, at least 80 pg/mL, at least 100 pg/mL, at least 150 pg/mL, at least 200 pg/mL, or at least 500 pg/mL.

As discussed in the foregoing and following description, methods and systems disclosed herein are intended to non-invasively detect a tissue or organ in a subject that is under duress as well as determine which disease or condition is affecting the tissue or organ under duress by detecting, quantifying, or otherwise analyzing at least one marker and at least one tissue-specific polynucleotide disclosed herein. In some cases, the at least one marker comprises a polynucleotide (e.g., cell-free polynucleotide) or a polypeptide. Some methods comprise detecting the polynucleotide or polypeptide by contacting the polynucleotide or polypeptide with at least one probe. In some cases, the at least one probe is only capable of binding to a wildtype version of the polynucleotide or polypeptide. In some cases, the at least one probe is only capable of binding to a mutant version of the polynucleotide or polypeptide. In some cases, for example, wherein the marker is a polynucleotide, detection comprises sequencing.

Some methods disclosed herein comprise isolating at least one marker and/or at least one tissue-specific polynucleotide. In some cases, the at least one marker and/or at least one tissue-specific polynucleotide comprise a cell-free polynucleotide. In some cases, isolating the cell-free polynucleotide comprises fractionating the sample from the subject. Some methods comprise removing intact cells from the sample. For example, some methods comprise centrifuging a blood sample and collecting the supernatant that is serum or plasma, or filtering the sample to remove cells. In some embodiments, cell-free polynucleotides are analyzed without fractionating the sample from the subject. For example, urine, cerebrospinal fluid or other fluids that contain little to no cells may not require fractionating. Some methods comprise sufficiently purifying the cell-free polynucleotides in order to detect, quantify, and/or analyze the cell-free polynucleotides. Various reagents, methods, and kits can be used to purify the cell-free polynucleotides. Reagents can include, but are not limited to, Trizol, phenol-chloroform, glycogen, sodium iodide, and guanidine resin. Kits include, but are not limited to, Thermo Fisher ChargeSwitch® Serum Kit, Qiagen RNeasy Kit, ZR serum DNA kit, Puregene DNA purification system, QIAamp DNA Blood Midi kit, QIAamp Circulating Nucleic Acid Kit, and QIAamp DNA Mini kit.

Some methods disclosed herein comprise enriching a sample for cell-free polynucleotides. For example, a sample of interest may contain RNA/DNA from bacteria. Some methods comprise exomal capture, thereby eliminating unwanted sequences and enriching the sample for polynucleotides of interest. In some cases, exomal capture comprises array-based capture or in-solution capture, fragments of DNA corresponding to RNAs of interest tethered to a surface or beads, respectively. Some methods also comprise filtering or removing other biological molecules or cells from the sample, such as proteins or platelets. In some instances, enriching the sample for cell-free polynucleotides includes preventing blood cell RNA contamination of a plasma sample. In some instances, using tubes free of EDTA prevents or reduces the presence of blood cell RNA in a plasma/serum sample.

Generally, methods disclosed herein comprise detecting or quantifying at least one marker and/or at least one tissue-specific polynucleotide. In some instances, quantifying and/or detecting the at least one marker and/or at least one tissue-specific polynucleotide comprises amplifying the at least one marker and/or at least one tissue-specific polynucleotide. In some cases involving cell-free RNA, quantifying and/or detecting the at least one marker and/or at least one tissue-specific polynucleotide comprises reverse transcribing the cell-free RNA. Any of a variety of processes can be employed to detect and/or quantify the marker or tissue-specific polynucleotide in a sample. In some cases involving cell-free, tissue-specific RNAs, RNA is isolated from a sample and reverse transcribed to produce cDNA prior to further manipulation, such as amplification and/or sequencing. In some embodiments, amplification is initiated at the 3′ end as well as randomly throughout the whole transcriptome in the sample to allow for amplification of both mRNA and non-polyadenylated transcripts. Suitable kits for amplifying cDNA include, for example, the Ovation® RNA-Seq System. Tissue-specific RNAs can be identified and quantified by a variety of techniques such as array hybridization, quantitative PCR, sequencing, etc.

Some methods disclosed herein comprise quantifying at least one marker and/or at least one tissue-specific polynucleotide described herein. In some cases, quantifying is useful for determining the severity of a condition. For example, some methods comprise comparing a quantity of marker and/or tissue-specific polynucleotide to a quantity of marker and/or tissue-specific polynucleotide in a first sample at a first time in the subject and quantifying the marker and/or tissue-specific polynucleotide in a second sample at a second time, wherein the subject was subjected to a therapy between the first time and the second time. Some methods comprise maintaining the therapy or changing the therapy (e.g., type, dose) based on information that resulted from the quantifying. Some methods comprise quantifying the marker and/or tissue-specific polynucleotide in additional samples at additional times, in between which the therapy is modulated.

Some methods of quantifying nucleic acids disclosed herein comprise sequencing at least one nucleic acid. Sequencing may be targeted sequencing. In some cases, targeted sequencing comprises specifically amplifying a select marker or a select tissue-specific polynucleotide disclosed herein and sequencing the amplification products. In some cases, targeted sequencing comprises specifically amplifying a subset of selected markers or a subset of select tissue-specific polynucleotides disclosed herein and sequencing the amplification products. Alternatively, some methods comprising targeting sequencing do not comprise amplifying the markers or tissue-specific polynucleotides. Some methods comprise untargeted sequencing. In some instances, untargeted sequencing comprises sequencing the amplification products, wherein a portion of the cell-free nucleic acids are not markers or tissue-specific polynucleotides. In some instances, untargeted sequencing comprises amplifying cell-free nucleic acids in a sample from the subject and sequencing the amplification products, wherein a portion of the cell-free nucleic acids are not markers or tissue-specific polynucleotides. In some instances, untargeted sequencing comprises amplifying cell-free nucleic acids comprising a marker or tissue-specific polynucleotide described herein. Sequencing may provide a number of reads that corresponds to a relative quantity of the marker or tissue-specific polynucleotide. In some instances, sequencing provides a number of reads that corresponds to an absolute quantity of the marker or tissue-specific polynucleotide. In some embodiments, the amplified cDNA is sequenced by whole transcriptome shotgun sequencing (also referred to as “RNA-Seq”). Whole transcriptome shotgun sequencing (RNA-Seq) can be accomplished using a variety of next-generation sequencing platforms such as the Illumina Genome Analyzer platform, ABI Solid Sequencing platform, or Life Science's 454 Sequencing platform. In some instances, identification of specific targets is performed by microarray, such as a peptide array or oligonucleotide array, in which an array of addressable binding elements specifically bind to corresponding targets, and a signal proportional to the degree of binding is used to determine quantity of the target in the sample. In some cases, the method of quantifying may include sequencing. In some instances, sequencing allows for parallel interrogation of thousands of genes without amplicon interference. In some instances, the method of quantifying may include quantitative PCR (qPCR). In some instances, there are so many control genes required to accurately quantify gene expression by qPCR, that quantifying with qPCR is inefficient. In other instances, sequencing efficiency and accurate quantification by sequencing may not be affected by the number of (control) genes analyzed. For at least the foregoing reasons, sequencing may be useful for some methods disclosed herein, wherein the health status of multiple organs (e.g., brain, heart, kidney, liver, etc.) is assessed.

Some methods of quantifying a nucleic acid disclosed herein comprise quantitative PCR (qPCR). In some instances, qPCR comprises a reverse transcription reaction of cell-free RNAs described herein to produce corresponding cDNAs. In some instances, cell-free RNA comprises a marker, a tissue-specific polynucleotide, and a cell-free RNA that is neither a marker nor a tissue-specific polynucleotide. Some cell-free RNA comprises a marker described herein, a tissue-specific polynucleotide described herein, and a cell-free RNA that is neither a marker nor a tissue-specific polynucleotide described herein. In some cases, qPCR comprises contacting the cDNAs that correspond to a marker, a tissue-specific polynucleotide, or a housekeeping gene (e.g., ACTB, ALB, GAPDH) with PCR primers specific to the marker, tissue-specific polynucleotide, or housekeeping gene.

Some methods disclosed herein comprise quantifying a blood cell-specific polynucleotide. Methods comprising qPCR disclosed herein may comprise contacting cDNA with primers corresponding to a blood cell-specific polynucleotide. Some blood cell-specific polynucleotides disclosed herein are nucleic acids that are predominantly expressed or even exclusively expressed by one or more types of blood cells. Types of blood cells can be generally categorized as white blood cells (also referred to as leukocytes), red blood cells (also referred to as erythrocytes), and platelets. In some instances, the blood cell-specific polynucleotide is used as a control in methods comprising quantifying tissue-specific polynucleotides and disease markers disclosed herein. In some cases, absence of an amplification product with primers corresponding to a blood cell-specific polynucleotide may be used to confirm the method is detecting cell-free RNAs in a blood, plasma, or serum sample and not RNA expressed in blood cells. By way of non-limiting example, blood-cell specific polynucleotides include polynucleotides expressed in white blood cells, platelets, or red blood cells, and combinations thereof. White blood cells include, but are not limited to lymphocytes, T-cells, B cells, dendritic cells, granulocytes, monocytes, and macrophages. By way of non-limiting example, the blood-specific polynucleotide may be encoded by a gene selected from CD4, TMSB4X, MPO, SOX6, HBA1, HBA2, HBB, DEFA4, GP1BA, CD19, AHSP, and ALAS2. The blood cell-specific polynucleotide may be encoded by CD4 and predominantly expressed by white blood cells. The blood cell-specific polynucleotide may be encoded by TMSB4X and expressed by multiple blood cell types (whole blood). The blood cell-specific polynucleotide may be encoded by MPO and predominantly expressed by neutrophil granulocytes. The blood cell-specific polynucleotide may be encoded by DEFA4 and predominantly expressed by neutrophils. The blood cell-specific polynucleotide may be encoded by GP1BA and predominantly expressed by platelets. The blood cell-specific polynucleotide may be encoded by CD19 and predominantly expressed by B cells. The blood cell-specific polynucleotide may be encoded by ALAS2, SOX6, HBA1, HBA2, or HBB and predominantly expressed by erythrocytes.

In some cases, the method of quantifying may be qPCR. qPCR may be a more sensitive method and therefore more accurately quantify RNA present at very low levels. In some instances, the method of quantifying may be sequencing. In some instances, sequencing requires more complex preparation of RNA samples and requires depletion or enrichment of nucleic acids in order to provide accurate quantification.

Often, methods disclosed herein comprise detecting or quantifying a combination of markers or a combination of tissue-specific polynucleotides. In some cases, a more conclusory diagnosis or assessment of the subject can be performed if multiple tissue-specific polynucleotides are detected. In some cases, the presence of each of the tissue-specific polynucleotides in a blood sample of the subject would not be indicative of damage to the tissue or origin of interest. However, their presence may collectively indicate damage to the tissue or origin of interest. Similarly, a more conclusory diagnosis or assessment of the subject can be performed if multiple markers are detected. In some cases, the presence of each of the markers in a blood sample of the subject would not be indicative of damage to the tissue or origin of interest. However, their presence may collectively indicate the condition in the tissue or origin of interest. The methods may comprise detecting or quantifying 2, 3, 4, 5, 6, 7, 8, 9, or 10 tissue-specific polynucleotides. The methods may comprise detecting or quantifying 2, 3, 4, 5, 6, 7, 8, 9, or 10 markers. Two or more of the markers may be known to interact in a common genetic pathway or common molecular signaling pathway. The common molecular signaling pathway may be a network of several proteins interacting to enact a cellular function, such as, by way of non-limiting example, an inflammatory response, apoptosis, cholesterol uptake, etc.

Similarly, in the case of cell-free DNAs, some methods disclosed herein employ tissue-specific modifications of DNA or chromatin to identify the tissue-specific polynucleotide in the sample. For example, a tissue-specific cell-free DNA may comprise a tissue-specific methylation pattern. A tissue-specific cell-free DNA may be complexed with a protein that is indicative of a specific tissue of origin (e.g., a transcription factor known to transcribe the gene in a particular tissue). Cell-free or circulating chromatin or chromatin fragments may have tissue-specific histone modifications (e.g., methylation, acetylation, and phosphorylation). In some of these cases, a method such as chromatin immunoprecipitation may be suitable for detecting/quantifying the tissue-specific polynucleotide. Cell-free tissue-specific DNA may be single-stranded or double-stranded DNA.

Some methods disclosed herein comprise use of a variety of methods of detecting the methylation pattern. Typically, the DNA will be subjected to a chemical conversion process that selectively modified either methylated or unmethylated nucleotides. For example, the DNA may be treated with bisulfite, which converts cytosine residues to uracil (which are converted to thymidine following PCR), but leaves 5-methylcytosine residues unaffected. Thus, bisulfite treatment introduces specific changes in the DNA sequence that depend on the methylation status of individual cytosine residues (“methylation-specific modification”), yielding single-nucleotide resolution information about the methylation status of a segment of DNA. Various analyses can be performed on the altered sequence to retrieve this information.

Some methods disclosed herein comprise subjecting DNA to oxidizing or reducing conditions prior to bisulfite treatment, so as to identify patterns of other epigenetic marks. For example, an oxidative bisulfite reaction can be performed. 5-methylcytosine and 5-hydroxymethylcytosine both read as a C in bisulfite sequencing. An oxidative bisulfite reaction allows for the discrimination between 5-methylcytosine and 5-hydroxymethylcytosine at single base resolution. Typically, the method employs a specific chemical oxidation of 5-hydroxymethylcytosine to 5-formylcytosine, which subsequently converts to uracil during bisulfite treatment. The only base that then reads as a C is 5-methylcytosine, giving a map of the true methylation status in the DNA sample. Levels of 5-hydroxymethylcytosine can also be quantified by measuring the difference between bisulfite and oxidative bisulfite sequencing. DNA may also be subjected to reducing conditions prior to bisulfite treatment. Reduction converts 5-formylcytosine residues in the sample nucleotide sequence into 5-hydroxymethylcytosine. As noted above, 5-formylcytosine converts to uracil upon bisulfite treatment, but 5-hydroxymethylcytosine does not. By comparing a first portion of a sample subjected to reductive bisulfite treatment to a second portion of a sample subjected to bisulfite treatment alone, locations of 5-formylcytosine marks can be identified.

As an alternative to inducing sequence changes based on methylation, methods disclosed herein may comprise inferring methylation status may by isolating or enriching polynucleotides comprising methylation, and identifying the methylated polynucleotides based on their sequences (e.g., by sequencing or probe hybridization). One process for enriching methylated sequences comprises modifying bases in a methylation-specific fashion, enriching for polynucleotides comprising the modification (e.g., by purification), and/or amplifying the enriched polynucleotides, and then identifying the polynucleotides. For example, 5-hydroxymethyl-modified cytosines (5hmC) may be selectively glycosylated in the presence of a UDP-glucose molecules and a beta-glucosyltransferase. The UDP-glucose molecules may comprise a label, such that the label becomes conjugated to the 5hmC-containing polynucleotide upon reaction with the UDP-glucose. The label can be a member of a binding pair (e.g., streptavidin/biotin or antigen/antibody), which allows isolation of modified fragments upon binding to the corresponding member of the binding pair. Isolated polynucleotides may be further enriched, such as in an amplification reaction (e.g., PCR), prior to identification.

Presence and/or quantity (relative or absolute) of a polynucleotide, as well as changes in sequence resulting from bisulfite treatment, can be detected using any suitable sequence detection method disclosed herein. Examples include, but are not limited to, probe hybridization, primer-directed amplification, and sequencing. Polynucleotides may be sequenced using any convenient low or high-throughput sequencing technique or platform, including Sanger sequencing, Solexa-Illumina sequencing, Ligation-based sequencing (SOLiD), pyrosequencing; strobe sequencing (SMR); and semiconductor array sequencing (Ion Torrent). The Illumina or Solexa sequencing is based on reversible dye-terminators. DNA molecules are typically attached to primers on a slide and amplified so that local clonal colonies are formed. Subsequently, one type of nucleotide at a time may be added, and non-incorporated nucleotides are washed away. Subsequently, images of the fluorescently labeled nucleotides may be taken and the dye is chemically removed from the DNA, allowing a next cycle. The Applied Biosystems' SOLiD technology employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. Another example of an envisaged sequencing method is pyrosequencing, in particular 454 pyrosequencing, e.g., based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed, and the cycle is repeated. Further examples of suitable sequencing techniques are sequencing by hybridization, sequencing by use of nanopores, microscopy-based sequencing techniques, microfluidic Sanger sequencing, or microchip-based sequencing methods. High-throughput sequencing platforms permit generation of multiple different sequencing reads in a single reaction vessel, such as 103, 104, 105, 106, 107, or more.

### Computer Control Systems

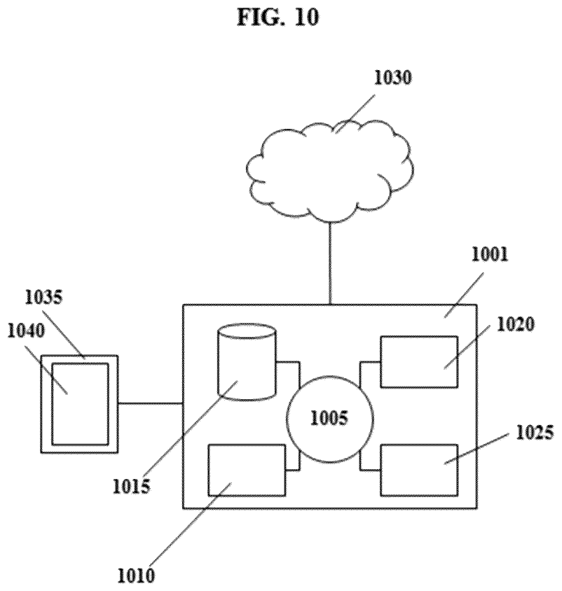

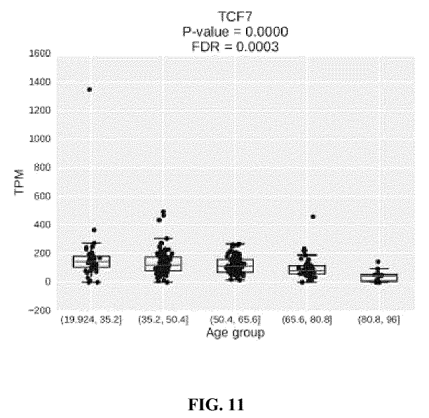

The present disclosure provides computer control systems that are programmed to implement methods of the disclosure. FIG. 10 shows a computer system 1001 that is programmed or otherwise configured to assess or detect AD in a subject. The computer system 1001 can regulate various aspects of the present disclosure, such as, for example, receiving or obtaining a biological sample; quantifying cell-free messenger RNA (cf-mRNA) levels of a plurality of cf-mRNAs in a biological sample, wherein said plurality of cell-free mRNAs corresponds to a first plurality of genes comprising KIAA0100, MAGI′, NNMT, MXD1, ZNF75A, SELL, ASS1, MNDA, and AC132217.4 or a second plurality of genes comprising SLU7, HNRNPA2B1, GGCt, NDUFA12, HSPB11, ATP6V1B2, SASS6, SUMO1, KRCC1, and LSM6; inputting said cf-mRNA levels to a classifier to obtain a risk score; generating a report based on the risk score; etc. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries, and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., a medical worker that is inquiring a risk score). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1140 for providing, for example, a report based on the risk score containing information direct to monitoring and/or treating AD progression. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, be used to generate the classifier to calculate a risk score of having AD or cognitive impairment.

### Kits

The present disclosure also provides kits. In some cases, a kit described herein comprises one or more compositions, reagents, and/or device components for measuring and/or detecting cf-mRNAs corresponding to one or more genes described herein. A kit as described herein can further comprise instructions for practicing any of the methods provided herein. The kits can further comprise reagents to enable the detection of the cf-mRNAs by various assays types such as reverse transcription, polynucleotide amplification, sequencing, probe hybridization, and microarray hybridization. Kits can also comprise a computer readable medium comprising computer executable code for implementing a method described herein.

In some embodiments, a kit provided herein comprises a plurality of oligonucleotide primers that hybridize to cDNA sequences transcribed from cf-mRNAs corresponding to a list of differentially expressed genes disclosed herein.

In some embodiments, kits described herein include a packaging material. As used herein, the term “packaging material” can refer to a physical structure housing the components of the kit. The packaging material can maintain sterility of the kit components, and can be made of material commonly used for such purposes (for example, paper, corrugated fiber, glass, plastic, foil, ampules, etc.). Kits can also include a buffering agent, a preservative, or a protein/nucleic acid stabilizing agent. Kits can include components for obtaining a biological sample from a patient. Non-limiting examples of such components can be gloves, hypodermic needles or syringes, tubing, tubes or vessels to hold the biological sample, sterilization components (e.g., isopropyl alcohol wipes or sterile gauze), and/or cooling material (e.g., freezer pack, dry ice, or ice). In some cases, kits disclosed herein are used in accordance of any of the disclosed methods.

Systems and kits can be provided herein to non-invasively detect a tissue or organ in a subject that is under duress as well as determine which disease or condition is affecting the tissue or organ under duress while taking into account changes in gene expression resultant of the aging process. Disclosed herein are kits for use in detecting a disease or condition in a subject, the kit comprising at least one reagent for detecting at least one marker, and at least one reagent for detecting at least one tissue-specific polynucleotide. Additionally or alternatively, the kits disclosed herein may be used to determine the location (e.g., tissue) and/or progression of a disease or condition in the subject. Additionally or alternatively, the kits disclosed herein may be used to determine if a therapy administered to the subject has affected the progression or stage of the disease or condition. Additionally or alternatively, the kits disclosed herein may be used to determine if a therapy administered to the subject has resulted in any unintended toxicity or side effects.

Provided herein are kits that comprise at least one reagent disclosed herein. The at least one reagent for detecting tissue-specific polynucleotides may comprise at least one reagent for detecting a cell-free polynucleotide. The at least one reagent for detecting at least one marker may comprise at least one reagent for a detecting cell-free polynucleotide. The at least one cell free polynucleotide may comprise cell-free DNA or cell-free RNA. The cell-free DNA may have a tissue-specific methylation pattern. The cell free polynucleotide may be a tissue-specific gene transcript. The at least one reagent for detecting at least one marker and/or the at least one reagent for detecting the tissue-specific polynucleotide may comprise a polynucleotide probe. The polynucleotide probe may bind to the cell-free polynucleotide. The polynucleotide probe may bind to the cell-free polynucleotide in a sequence-dependent manner. The polynucleotide probe may bind to a cell-free polynucleotide corresponding to a wildtype version of a gene, but not a mutant version of the gene. Alternatively, the polynucleotide probe may bind to a cell-free polynucleotide corresponding to a mutant version of a gene, but not a wildtype version of the gene. The polynucleotide probe may be attached to a signaling moiety. By way of non-limiting example, the signaling moiety may be selected from a hapten, a fluorescent molecule, and a radioactive isotope. The kit may be specific for one disease or condition. The kit may comprise as few as 1, 2, 3, 4, or 5 polynucleotide probes in order to detect a disease or condition in a subject. The kit may be specific for multiple diseases or conditions. The kit may comprise 5 to 10, 10 to 20, 10 to 100, 10 to 1000, 100 to 1000, 100 to 10,000, or more 4 polynucleotide probes.

Provided herein are kits that comprise at least one reagent disclosed herein. The at least one reagent for detecting at least one marker and/or the at least one reagent for detecting the tissue-specific polynucleotide may comprise a primer. The primer may be a reverse transcriptase primer. The primer may be a PCR primer. The primer may amplify the at least one marker, at least one tissue-specific polynucleotide, or portions thereof. The primer may amplify the cell-free polynucleotide in a sequence-dependent manner. The primer may amplify a cell-free polynucleotide or portion thereof corresponding to a wildtype version of a gene, but not a mutant version of the gene. Alternatively, the primer may amplify a cell-free polynucleotide or portion thereof corresponding to a mutant version of a gene, but not a wildtype version of the gene. The kit may further comprise an amplification reporter that provides a user of the kit with the quantity of the at least one marker and/or the at least one reagent for detecting the tissue-specific polynucleotides. Typically, the quantity is a relative quantity based on a reference sample. The amplification signaling reagent may be selected from intercalating fluorochromes or dyes. The amplification signaling reagent may be SYBR Green.

Provided herein are kits that comprise at least one reagent disclosed herein. The at least one reagent for detecting at least one marker and/or the at least one reagent for detecting the tissue-specific polynucleotide may comprise a peptide that binds to the at least one marker or tissue-specific polynucleotide. The peptide may be part of an antibody, or a polynucleotide binding protein (e.g., transcription factor, histone). The at least one reagent for detecting at least one marker and/or the at least one reagent for detecting the tissue-specific polynucleotide may comprise a signaling moiety that emits a signal, wherein the signal being emitted or lost is indicative of a presence or a quantity of a marker or a tissue-specific polynucleotide. Examples of signaling moieties include, but are not limited to, dyes, fluorophores, enzymes, and radioactive particles. The at least one reagent may further comprise a signaling moiety detector for detecting the signal or absence thereof.

Disclosed herein are kits for use in detecting whether or not a tissue or organ is affected by a condition, wherein the kits comprise at least one probe or primer for a marker of the condition. Further disclosed herein are kits for use in detecting the location of a tumor, pathogen or disease, wherein the kits comprise at least one probe or primer for a marker of the condition. In some instances, the kits comprise at least one probe and at least one primer. In some instances, the marker is a polynucleotide and the primer or probe is a polynucleotide that hybridizes to a target of interest. In some instances, the marker is a peptide or protein and the probe is an antibody or antibody fragment capable of binding the peptide or protein. In some instances, the probe is a small molecule that binds to the marker. In some instances, the probe is conjugated to a tag that can be used to retrieve the marker, quantify the marker or detect the marker. The at least one condition or disease may be at least one of: inflammation, apoptosis, necrosis, fibrosis, infection, autoimmune disease, arthritis, liver disease, neurodegenerative disease, and cancer.

Disclosed herein are kits for use in detecting a disease or condition in a subject, the kit comprising at least one reagent for detecting at least one marker, and at least one reagent for detecting at least one tissue-specific polynucleotide. The kit may further comprise a solid support, wherein the polynucleotide probe, the primer and/or the peptide is attached to a solid support. The solid support may be selected from a bead, a chip, a gel, a particle, a well, a column, a tube, a probe, a slide, a membrane, and a matrix.

Disclosed herein are kits for use in detecting a disease or condition in a subject, the kit comprising at least one reagent for detecting at least one marker, and at least one reagent for detecting at least one tissue-specific polynucleotide. Two or more components of the kits disclosed herein may be separate. Two or more components of the kits disclosed herein may be integrated. Two or more components of the kits disclosed herein may be integrated into a device. The device may allow for a user to simply add at least one sample from the subject to the device and receive a result indicating whether or not the subject has the disease or condition and/or which tissue(s) of the subject is affected by the disease or condition. In some cases, the user may add at least one reagent to the device. In other cases, the user does not have to add any reagents to the device.

Disclosed herein are kits for use in detecting a disease or condition in a subject, the kit comprising at least one reagent for detecting at least one marker, and at least one reagent for detecting at least one tissue-specific polynucleotide. The at least one tissue-specific polynucleotide or marker may comprise a cell free polynucleotide. The at least one marker may comprise RNA. The at least one tissue-specific polynucleotide may comprise at least one tissue-specific RNA, wherein a tissue-specific RNA is an RNA expressed only in a specific tissue or at a level in a specific tissue that is substantially higher than the level at which it is expressed in other tissues. For example, a tissue-specific gene may be a gene for which expression in a particular tissue or group of tissues is at least 2-fold, 5-fold, 10-fold, or 25-fold greater than any other tissue or group of tissues (e.g., any individually, or all other tissues or group of tissues combined). The at least one tissue-specific polynucleotide or marker may comprise at least one tissue-specific methylated DNA, wherein the tissue-specific methylated DNA comprises a tissue-specific methylation pattern. Alternatively or additionally, the tissue-specific methylated DNA may comprise DNA with a methylation pattern that occurs in only one tissue or at a level in a tissue that is substantially higher than the level at which it occurs in other tissues. The tissue may be determined to be damaged by the condition if: (a) the level of at least one of the marker is above the reference level of the at least one marker and (b) the level of at least one of the tissue-specific polynucleotide is above the reference level of the at least one tissue-specific polynucleotide. The at least one tissue-specific polynucleotide may comprise two or more polynucleotides each of which is specific for a different tissue (e.g., 2, 3, 4, 5, 10, 15, 25, or more different tissues). The tissue may be at least one of: whole blood, bone, epithelium, hypothalamus, smooth muscle, lung, thymus, lymph node, thyroid, heart, kidney, brain, cerebellum, liver, and skin. The marker and/or tissue-specific polynucleotide may correspond to a gene. In general, a marker or tissue-specific polynucleotide “corresponds to a gene” if it is a DNA molecule comprising the gene (or an identifiable portion thereof), or is an expression product of the gene (e.g., an RNA transcript or a protein product).

Further disclosed herein are systems for carrying out methods of the present disclosure. In general, a system may comprise various units capable of performing the steps of methods disclosed herein, for example, a sample processing unit, an amplification unit, a sequencing unit, a detection unit, a quantifying unit, a comparing unit, and/or a reporting unit. In some embodiments, the system comprises: a memory unit configured to store results of: (i) an assay for detecting at least one marker of at least one condition in a first sample of a subject and (ii) an assay for detecting at least one tissue-specific RNA in a second sample of a subject, wherein the at least one tissue-specific RNA is a cell-free RNA specific to a tissue; at least one processors programmed to: (i) quantify a level of the at least one marker; (ii) quantify a level of the at least one tissue-specific polynucleotide; (iii) compare the level of the at least one marker to a corresponding reference level of the marker; (iv) compare the level of the at least one tissue-specific polynucleotide to a corresponding age-dependent reference level of the tissue-specific polynucleotide; and (v) determine presence of or relative change in damage of the tissue by the at least one condition based on the comparing; and an output unit that delivers a report to a recipient, wherein the report provides results of step (b). The system may provide a recommendation for medical action based on the results of step (b). The medical action may comprise a treatment. The first sample and the second sample may be the same. The first sample and the second sample may be different. The first sample and the second sample may be different in that they were obtained at different times. The first sample and the second sample may be different in that they are different fluids. The first and/or second sample may be a fluid selected from the group consisting of: blood, a blood fraction, saliva, sputum, urine, semen, a transvaginal fluid, a cerebrospinal fluid, sweat, or a breast fluid. The first and/or second sample may be plasma.

The systems disclosed herein may be used with any one of the kits or devices disclosed herein. The systems may be integrated with any one of the kits or devices disclosed herein. The devices disclosed herein may comprise any one of the systems disclosed herein. In some embodiments, the system comprises a computer system. A computer for use in the system may comprise at least one processor. Processors may be associated with at least one controller, calculation unit, and/or other unit of a computer system, or implanted in firmware as desired. If implemented in software, the routines may be stored in any computer readable memory such as in RAM, ROM, flashes memory, a magnetic disk, a laser disk, or other suitable storage medium. Likewise, this software may be delivered to a computing device via any known delivery method including, for example, over a communication channel such as a telephone line, the Internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers can rely on server computers for resources, such as files, devices, and even processing power. In some embodiments, the server computer handles all of the database functionality. The client computer can have software that handles all the front-end data management and can also receive data input from users.

Systems disclosed herein may be configured to receive a user request to perform a detection reaction on a sample. The user request may be direct or indirect. Examples of direct request include those transmitted by way of an input device, such as a keyboard, mouse, or touch screen. Examples of indirect requests include transmission via a communication medium, such as over the internet (either wired or wireless).

Systems disclosed herein may further comprise a report generator that sends a report to a recipient, wherein the report contains results of a method described herein. A report may be generated in real-time, such as during a sequencing read or while sequencing data is being analyzed, with periodic updates as the process progresses. In addition, or alternatively, a report may be generated at the conclusion of the analysis. In some embodiments, the report is generated in response to instructions from a user. In addition to the results of detection or comparison, a report may also contain an analysis, conclusion or recommendation based on such results. For example, markers associated with a disease or condition are detected and levels of a tissue-specific polynucleotide are above a normal range, the report may include information concerning this association, such as a likelihood that subject has the disease or condition, which tissues are or are not affected, and optionally a suggestion based on this information (e.g., additional tests, monitoring, or remedial measures). The report can take any of a variety of forms. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., at least one computers and/or at least one servers).

The disclosure provides a computer-readable medium comprising code that, upon execution by at least one processor, implements a method of the present disclosure. A machine readable medium comprising computer-executable code may take many forms, including but not limited to, a tangible storage medium, a carrier wave medium or physical transmission medium. Non-volatile storage media include, for example, optical or magnetic disks, such as any of the storage devices in any computers) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying at least one sequence of at least one instruction to a processor for execution.

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in the context of a number refers to a range spanning from 10% greater than the number to 10% less than the number.

As used herein, the phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone; B alone; C alone; A and B together; A and C together; B and C together; or A, B, and C together.

The terms “determining,” “measuring,” “evaluating,” “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “panel,” “biomarker panel,” “protein panel,” “classifier model,” and “model” are used interchangeably herein to refer to a set of biomarkers, wherein the set of biomarkers comprises at least two biomarkers. Exemplary biomarkers are cf-mRNAs mapped to a list of differentially expressed genes disclosed herein. However, additional biomarkers are also contemplated, for example, age or gender of the individual providing a sample. The biomarker panel is often predictive and/or informative of a subject's health status, disease, or condition.

The “level” of a biomarker panel refers to the absolute and relative levels of the panel's constituent markers and the relative pattern of the panel's constituent biomarkers.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cognitive impairment. The cognitive impairment can be a symptom for AD. In some cases, the subject is not necessarily diagnosed or suspected of being at high risk for the disease.

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e., patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “machine learning,” “machine learning procedure,” “machine learning operation,” and “machine learning algorithm” generally refer to any system or analytical and/or statistical procedure that may progressively improve computer performance of a task. Machine learning may include a machine learning algorithm. The machine learning algorithm may be a trained algorithm. Machine learning (ML) may comprise one or more supervised, semi-supervised, or unsupervised machine learning techniques. For example, an ML algorithm may be a trained algorithm that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naive Bayes, Gaussian naive Bayes, multinomial naive Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

### EXAMPLES

The following illustrative examples are representative of embodiments of the compositions and methods described herein and are not meant to be limiting in any way.

### Example 1—Clinical Specimens

A total of 242 plasma specimens, comprising 126 Alzheimer's disease patients and 116 age matched controls, from five independent patient cohorts of AD and NCIs were examined. These cohorts included: University of California San Diego, University of Kentucky, University of Washington St Louis, GEMS (Indiana), and BioIVT. The detailed patient demographics and clinicopathological characteristics are shown in Table 2. Written informed consent was obtained from all patients, and the study was approved by the institutional review boards of all the participating institutions.

TABLE 2
Overall patient characteristics

| Variable | | AD | NCI |
|---|---|---|---|
| Patient number | | 125 | 116 |
| Age | Average (±SEM) | 75.0 ± 0.9 | 75.9 ± 0.9 |
| Sex | Female (%) | 74 (59%) | 67 (58%) |
| | Male (%) | 51 (41%) | 54 (42%) |
| Cognitive impairment | MMSE (patient number) | 125 | 62 |
| test | CDR (patient number) | 66 | 76 |

All clinical diagnoses were performed according to the NINCDS-ADRDA (Criteria of National Institute of Neurological and Communicative Disorders and Stroke and the Alzheimer's Disease and Related Disorders Association) and according to the recommendations from the National Institute of Ageing—Alzheimer's Association workgroups on diagnostic guidelines for Alzheimer's disease.

### Example 2—RNA Extraction, Library Preparation, and Whole-Transcriptome RNA-Seq

RNA was extracted from up to 1 mL of plasma using QIA amp Circulating Nucleic Acid Kit (Qiagen) and eluted in 15 μl volume. ERCC RNA Spike-In Mix (Thermo Fisher Scientific, Cat. #4456740) was added to RNA as an exogenous spike-in control according to manufacturer's instruction (Ambion). Agilent RNA 6000 Pico chip (Agilent Technologies, Cat. #5067-1513) was used to assess the integrity of extracted RNA. RNA samples were converted into a sequencing library. Qualitative and quantitative analysis of the NGS library preparation process was conducted using a chip-based electrophoresis and libraries were quantified using a qPCR-based quantification kit. Sequencing was performed using Illumina NextSeq500 platform (Illumina Inc.), using paired-end sequencing, 75-cycle sequencing. Base-calling was performed on an Illumina BaseSpace platform (Illumina Inc.), using the FASTQ Generation Application. For sequencing data analysis, adaptor sequences were removed, and low-quality bases were trimmed using cutadapt (v1.11). Reads shorter than 15 base-pairs were excluded from subsequent analysis. Read sequences greater than 15 base-pairs were compared to the human reference genome GRCh38 using STAR (v2.5.2b) with GENCODE v24 gene models. Duplicated reads were removed using the samtools (v1.3.1) rmdup command. Gene-expression levels were calculated from de-duplicated BAM files using RSEM (v1.3.0).

Differential expression analysis was implemented with DESeq2 (v1.12.4) using read counts as input. Genes with fewer than 250 total reads across the entire cohort were excluded from subsequent analysis. Technical replicates were averaged and combined before the DE analysis.

Samples were obtained from five different sources described in Table 3. In order to correct for batch-effects associated with sample sources, a multi-factor model “˜source+disease status,” was implemented including sample source as a potential confounder. The batch correction was effective as indicated by the PCA plot after correction. Benjamin-Hochberg correction was used to correct for multiple testing and obtain adjusted p-values (FDR cutoff of 0.05 was used to select dysregulated genes).

Pathway enrichment analysis was conducted using Ingenuity Pathway Analysis (IPA) software version 47547484. Complete list of differentially expressed and genes correlated with MMSE and CDR were uploaded to IPA and Expression Analysis was used to determine pathways that are highly enriched. IPA categories including: Canonical pathways and “Top diseases and bio functions” were examined.

### Example 3—Brain-Specific Gene Establishment

Genes that show substantially higher expression in a particular tissue (cell-type) compared to other tissue types (cell-types) are considered tissue (cell-type) specific genes, such as brain-specific genes. Tissue (cell-type) transcriptome expression levels were obtained from the following two public databases: GTEx (www_gtexportal_org/home/) for gene expression across 51 human tissues and Blueprint Epigenome (www_blueprint-epigenome_eu/) for gene expression across 56 human hematopoietic cell types. For each individual gene, the tissues (cell-types) were ranked by their expression of that particular gene and if the expression in the top tissue (cell-type) is greater than about 20-fold higher than all the other tissues (cell-types) the gene was considered specific to the top tissue (cell-type).

### Example 4—Bioinformatic Analysis/Classifier Development

To build a gene-expression classifier, the cohort was split to 65% and 35%, with first 65% assigned as the “training cohort” and the second 35% as the “validation cohort.” These gene-expression values and logistic regression models with ridge regularization were applied to identify AD samples. Logistic regression analysis with L1 regularization within the scikit-learn Python library was used for implementation of the classification. Meta-parameters are determined by cross-validation performed 15 times, by randomly withholding 40% of the samples for validation within the “training cohort.”

In order to ensure an unbiased evaluation of classifier performance, the samples sourced from University of Kentucky were used as “training cohort” and samples from all other sources as “validation cohort.” None of the samples in the validation cohort was used in any way during model training. At the feature selection step, DESeq2 was run on the training cohort and the top 1,476 genes differentially expressed between AD and NCI samples were selected. The expression levels (TPM) of those 1,658 genes were then used in the subsequent training of the classifiers. The training of the classifiers was implemented using Python library Scikit-learn (scikit-learn_org/stable/, v0.20.1). Logistic regression, random forest, support vector machine (SVM), K-nearest neighbors classifiers were implemented with classes sklearn.linear model.LogisticRegression, sklearn.ensemble.RandomForestClassifier, sklearn.svm.SVC, and sklearn.neighbors.KNeighborsClassifier, respectively. Meta-parameters were determined by 15-fold cross-validation on the training cohort. Next, the trained classifiers were applied to the validation cohort and the predicted risk score was obtained for each sample in the validation cohort. By comparing the risk score with the true disease status of the samples the receiver-operating-characteristic (ROC) curves were able to be plotted and the area under the curve (AUCs) were calculated. Confidence intervals for the ROC curves were calculated according to DeLong.

A normalization was first implemented whereby the expression levels of each gene were divided by its maximum value across the samples. This step is designed to rescale the expression levels among different genes so as to avoid a few highly expressed genes dominating the decomposition process. The normalized expression matrix was then subject to NMF decomposition using sklearn.decomposition.NMF within the Python library Scikit-learn (//scikit-learn_org/stable/). NMF decomposition achieves a more parsimonious representation of the data by decomposing expression matrix into the product of two matrices X=WH. X is the expression matrix with n rows (n samples) and m columns (m genes); W is the coefficient matrix with n rows (n samples) and p columns (p components); H is the loading matrix with p rows (p components) and m columns (m genes). W is in a sense a summarization of the original matrix H with reduced number of dimensions. H contains information about how much each gene contribute to the components. Biological interpretation of the derived components was achieved by performing pathway analysis on the top genes that contribute the most to each component. Patient grouping was conducted by performing hierarchical clustering on the coefficient matrix W. Hierarchical clustering was implemented using Python library SciPy (v1.3.0) class scipy.cluster.hierarchy.linkage with parameters method=“average” and metric=“correlation.”

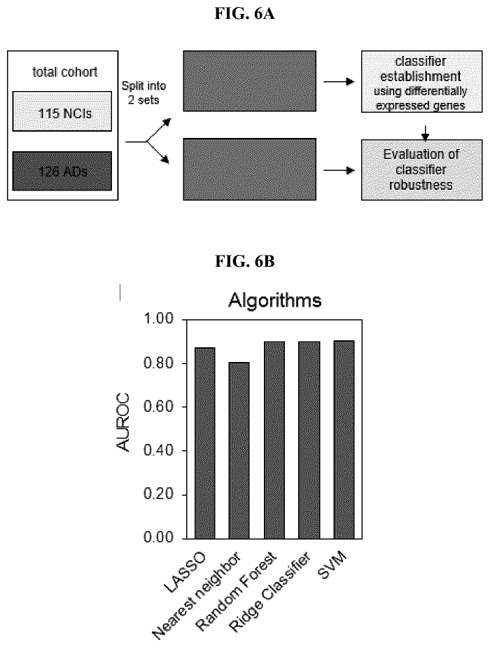

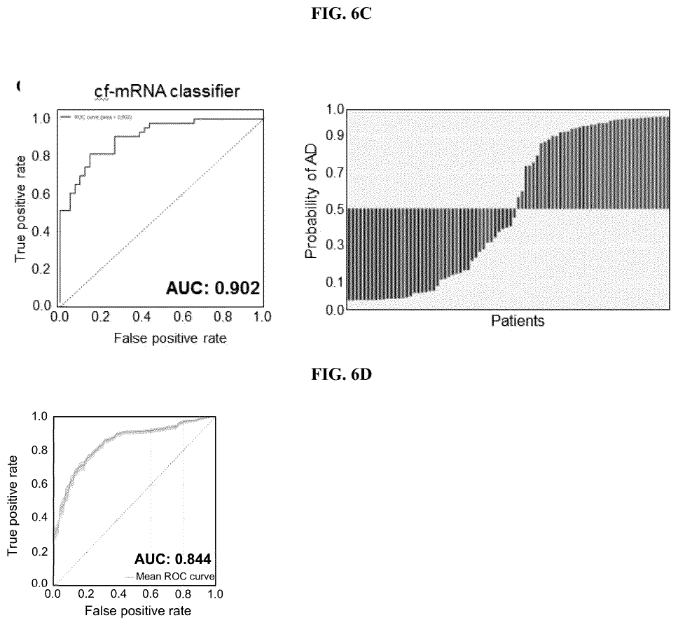

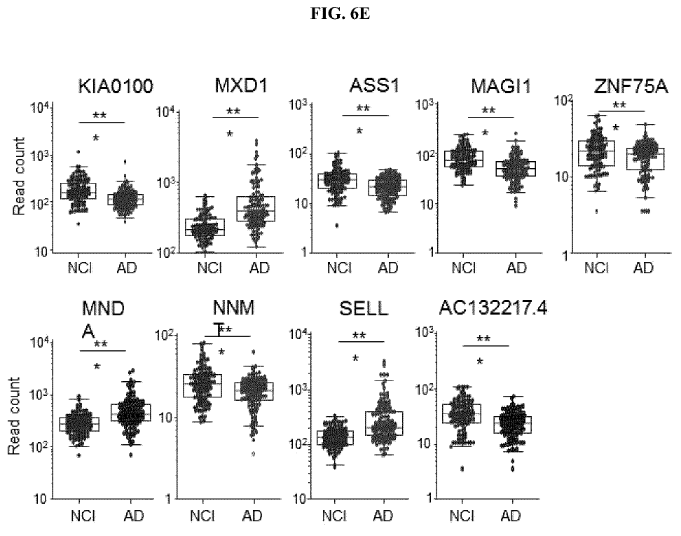

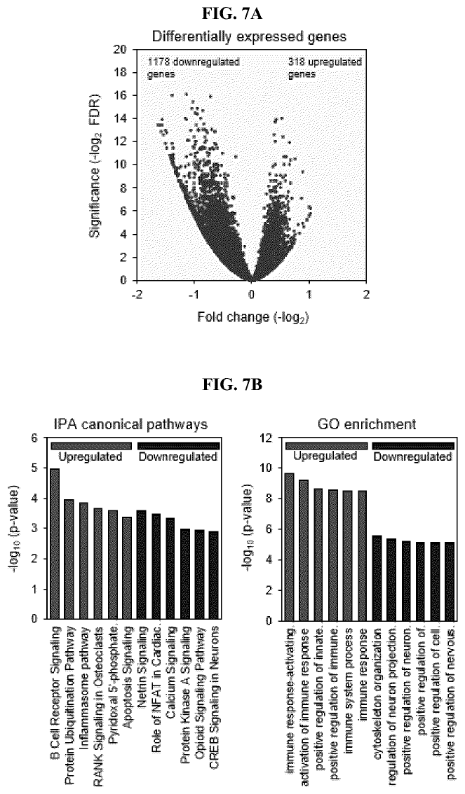

To ensure that classifier performance is assessed unbiasedly, a classifier is first built using exclusively samples from University of Kentucky (control n=24, AD n=66) (FIG. 6A). Differentially expressed genes identified in this University of Kentucky (UKy) only cohort (1,658 genes with FDR<0.05) were selected as input features for the classifier. This set of genes significantly overlap with the 2,591 dysregulated genes identified using the entire cohort (i.e., 942 out of the 1,094 down-regulated genes identified using UKy cohort overlap with those identified using the entire cohort, p-value<10e-8; 451 out of the 564 up-regulated genes identified using UKy cohort overlap with those identified using the entire cohort, p-value<10e-8; hypergeometric test). The classifier model was then tested on the testing set comprised of the remainder of the AD (n=60) and control samples (n=92) derived from four independent sources. Classification performance evaluated by calculating AUROC (Area Under the Receiver Operating Characteristics) in the testing cohort was AUROC: 0.83 (95% CI: 0.77-0.89) (FIG. 6B). Youden Index was used to establish the cutoff at 0.868 with a sensitivity of 83.3 (95% CI: 71.5-91.7%), specificity of 68.5 (95% CI: 58.0-77.8%).

### Example 5—Statistical Analysis

Risk scores derived from the gene-classifier multivariate logistic regression model were used to plot receiver-operating-characteristic (ROC) curves and calculate area under the curves (AUCs). Area under the ROC curve (AUC) is calculated for each of the 15 iterations of cross-validation. Average ROC curves are calculated from these 15 cross-validations. Confidence intervals for the ROC curves were calculated using the method of DeLong. Pearson's correlation analysis was used to examine correlation between two variables. Student's t-test was used to evaluate the difference between two variables. All statistical analyses were performed using R (3.3.3, R Development Core Team, //cran_r-project_org/) and MedCalc statistical software version 19 (MedCalc Software bvba, Ostend, Belgium).

### Example 6—Robust Characterization of Cf-RNA Transcriptome Using Low In-Put Plasma RNA

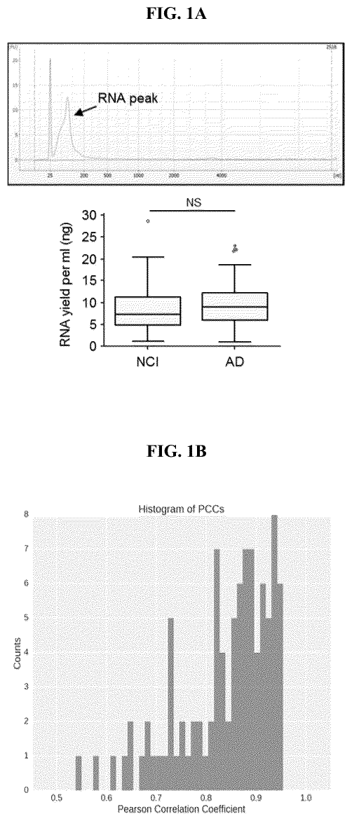

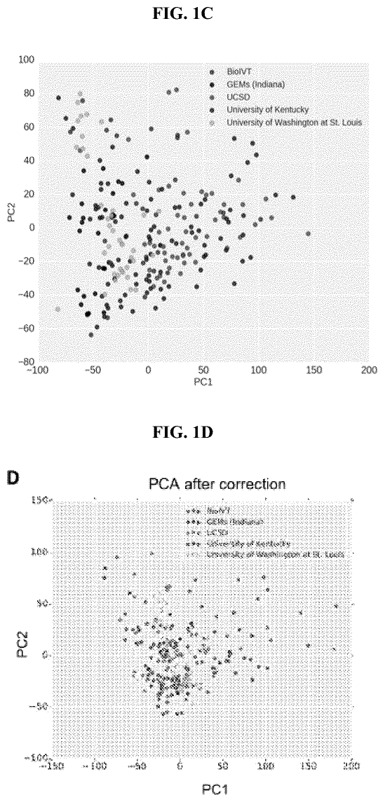

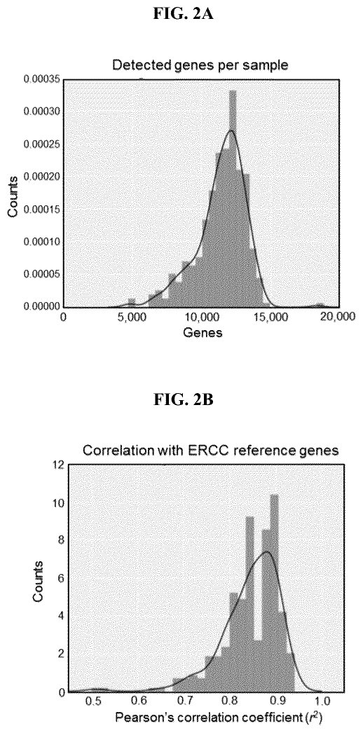

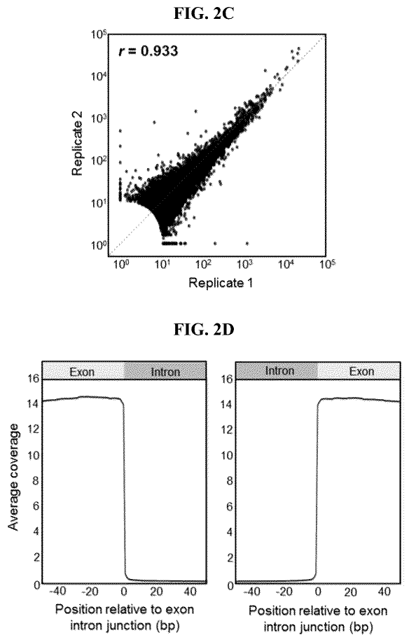

RNA extracted from 400 μm to 1 ml of plasma obtained from 126 patients with AD and 116 age-matched controls were sequenced. The average plasma cf-RNA yield did not differ between AD and NCI controls (8.55 and 9.55 ng respectively) (FIG. 1A). Following sequencing runs, the average protein-coding genes identified were 11,714 (transcripts detected at >5 TPM) (FIG. 2A). Using external RNA spike-in mix controls, ERCC (External RNA Controls Consortium), the accuracy of the present protocol was confirmed with the observed levels of ERCC transcripts correlated with the expected spiked-in copy numbers (mean r=0.92, FIG. 2B). In addition, comparison of the transcript levels between technical replicates in 96 samples correlated tightly (mean r=0.87), highlighting robust technical reproducibility of the protocol (FIGS. 2C and 1B). Finally, the read distribution across exon-intron splice junctions showed that DNA contamination was negligible (FIG. 2D). Together these results demonstrate reliable technical performance of the cf-mRNA sequencing protocol for generating diverse, quantitative, and reproducible sequencing data regardless of the AD status of the patient.

### Example 7—Identification of Alzheimer's Disease Associated Cf-mRNA Gene-Expression Profile

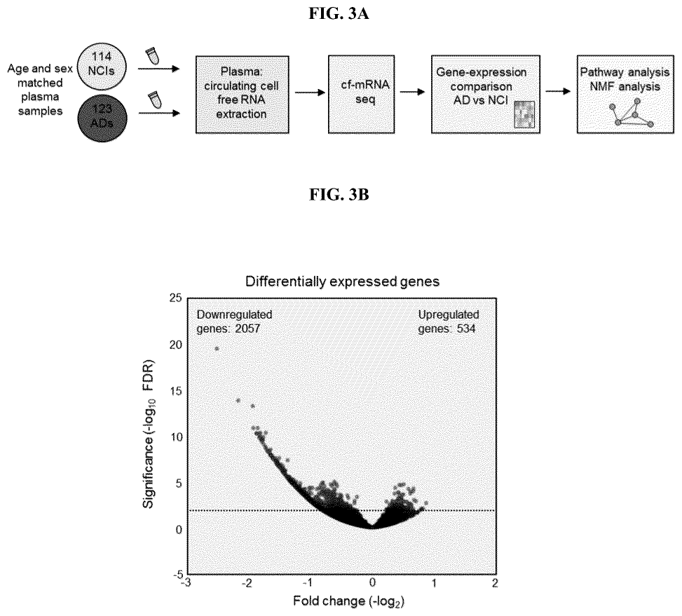

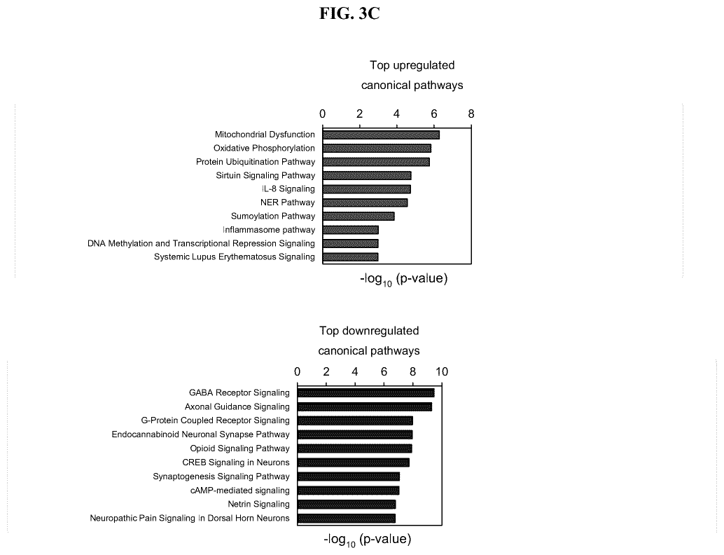

To identify differences in the circulating transcriptome between AD patients and controls, cf-mRNA isolated from 241 plasma samples, comprised of 126 AD patients and 115 age-matched NCI controls, from five independent sources, were sequenced (FIG. 3A; see Tables 2 and 3 for participant characteristics).

TABLE 3
Cohort characteristics

| | | AD | | NCI | | | | |
|---|---|---|---|---|---|---|---|---|
| | | | U of | UW St. | U of | GEMS | | |
| Variable | | UCSD | Kentucky | Louis | Kentucky | (Indiana) | BioIVT | Total |
| Patient number | | 59 | 66 | 39 | 23 | 26 | 28 | 241 |
| Sex | Female | 37 | 37 | 23 | 14 | 14 | 16 | 141 |
| | Male | 22 | 29 | 16 | 9 | 12 | 12 | 100 |
| Age (average ± | Overall | 74.2 ± 1.1 | 77.2 ± 1.5 | 72.3 ± 0.7 | 83.9 ± 1.4 | 80.2 ± 0.6 | 73.1 ± 1.8 | 75.5 ± 0.6 |
| SEM) | Female | 74.2 ± 1.4 | 77.2 ± 2.1 | 72.3 ± 0.9 | 84 ± 1.9 | 80.2 ± 0.9 | 73.7 ± 2.4 | 75.5 ± 0.8 |
| | Male | 74.6 ± 1.6 | 76.5 ± 2.3 | 72.1 ± 1.1 | 83.7 ± 2.4 | 80.3 ± 1.0 | 72.0 ± 2.3 | 75.8 ± 1.0 |
| MMSE (average ± | Overall | 20.5 ± 0.6 | 19.0 ± 1.0 | 29.4 ± 0.1 | 26.5 ± 1.1 | — | — | — |
| SEM) | Female | 20.5 ± 0.7 | 19.0 ± 1.4 | 29.4 ± 0.1 | 26.4 ± 1.4 | — | — | — |
| | Male | 20.6 ± 1.6 | 18.8 ± 1.6 | 29.4 ± 0.2 | 26.3 ± 1.9 | — | — | — |
| CDR | Overall | — | 1.16 ± 0.12 | 0 | 0 | — | — | — |
| | Female | — | 1.16 ± 0.16 | 0 | 0 | — | — | — |
| | Male | — | 1.16 ± 0.18 | 0 | 0 | — | — | — |

2,591 differentially expressed genes were identified between AD and NCI (FDR<0.05, FIG. 3B), of which 2,057 transcripts were downregulated while 534 transcripts were upregulated in the circulation of AD patients. The terms “upregulated” and “downregulated” were used to describe changes in the number of transcripts in the circulation of AD patients compared to NCI controls. To evaluate the functional roles of these differentially expressed genes, IPA pathway analysis was used to determine the pathways and biological processes that are most affected by AD. IPA analysis revealed that many of the canonical pathways that are identified by downregulated transcripts of AD patients were associated with neuronal signaling pathways including: GABA receptor signaling, netrin signaling, synaptic long-term depression and opioid signaling pathway, while upregulated transcripts were enriched in canonical pathways that are associated with immune response (e.g., IL-8 signaling, inflammasome, and neuroinflammation signaling pathway), mitochondrial activity (e.g., sirtuin signaling pathway and mitochondrial dysfunction) and proteostasis (e.g., sumoylation). The top canonical pathways identified using the transcripts downregulated in AD patients were associated with neuronal functions, including GABA receptor signaling, CREB signaling in neurons, netrin signaling and synaptogenesis signaling pathway, among others (FIG. 3C).

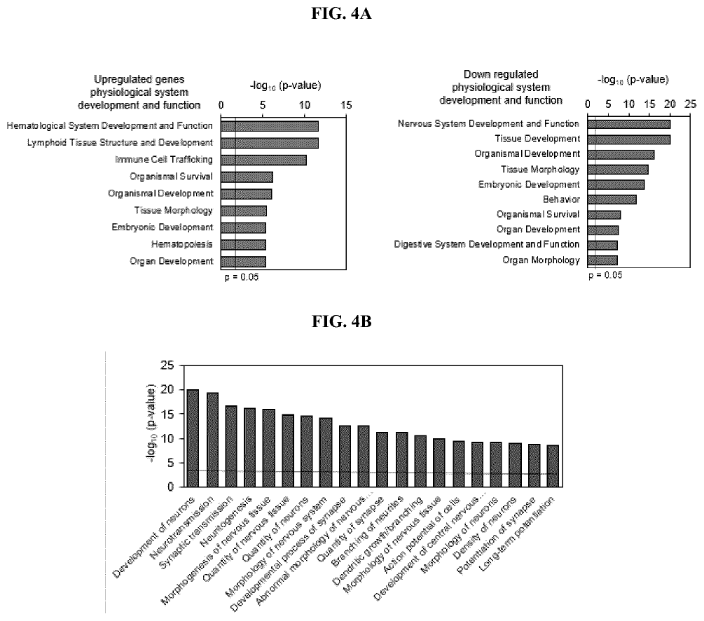

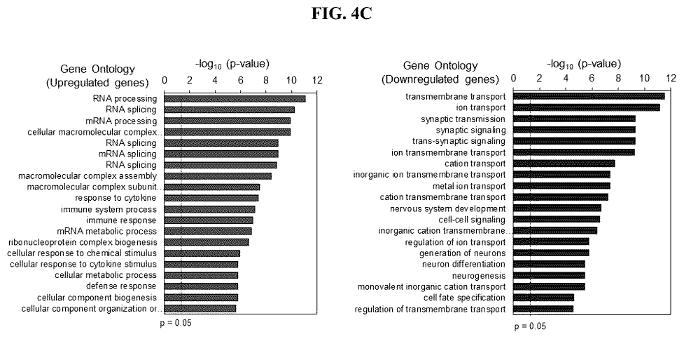

Further, using IPA analysis, the biological processes that are dysregulated in AD were examined. Consistent with the canonical pathway analysis, upregulated transcripts in AD patients were significantly enriched in pathways associated with immune response activation (e.g., IL-8 signaling and inflammasome pathway), mitochondrial activity (e.g., mitochondrial dysfunction, oxidative phosphorylation and sirtuin signaling pathway) and proteostasis (e.g., sumoylation, protein ubiquitination and unfolded protein response) (FIG. 3C). Regarding the biological processes, genes downregulated in AD patients were enriched in “nervous system development and function” category. Biological processes which are associated with the loss of neurons and synapse including “development of neurons,” “neurotransmission,” and “synaptic transmission” were the most significantly enriched terms, indicating overall decline of neurons and synaptic connections associated transcripts in the cf-mRNA transcriptome of AD patients (FIG. 4B). Consistently, it was observed that a significant portion of genes downregulated in cf-mRNA of AD patients were brain specific-genes (p=6.17×10⁻¹⁰, FIG. 4A). Last, Gene Ontology enrichment analysis confirmed that the genes that are downregulated in AD patients are associated with neuronal function, while upregulated genes are enriched in immune response and RNA splicing related processes, all consistent with AD pathophysiology (FIG. 4C).

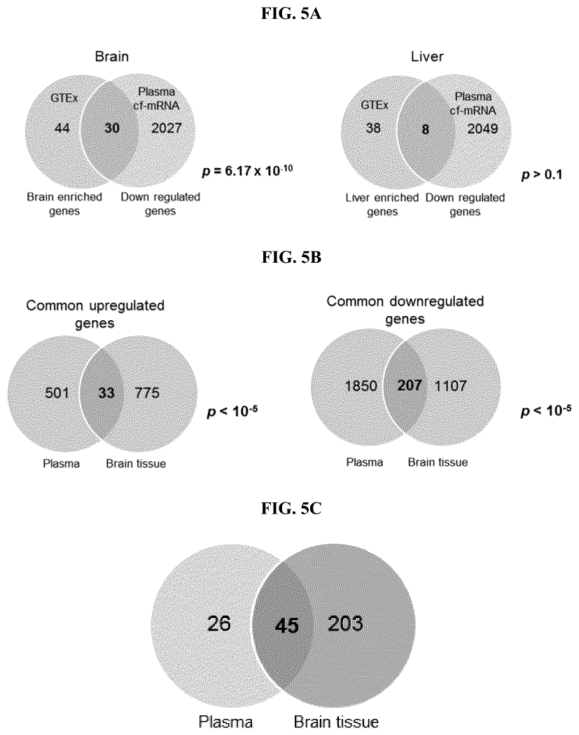

In addition, a portion of brain-specific genes were downregulated in cf-mRNA of AD patients (p=6.17×10⁻¹⁰, FIG. 5A). To further ascertain that AD-associated transcriptional alterations in cf-mRNA corresponded to the gene-expression changes in the brain tissues, the differentially expressed genes identified in AD cf-mRNA were compared with a previous RNA-seq dataset which examined transcriptional changes in the hippocampal autopsy tissues (FIG. 5B). An overlap between the differentially expressed genes of AD and NCI was observed in the brain tissue against those of cf-mRNA for both up and down-regulated genes (both p<10-5). Furthermore, there was overlap of identified pathways between cf-mRNA and brain tissues (FIG. 5C). These data collectively support that cf-mRNA transcriptome captures transcriptional changes associated with AD.

### Example 8—Robust Classification of Alzheimer's Disease Patients Versus Non-Cognitively Impaired Controls Based on Cf-mRNA Profile

Machine learning algorithms were used to build cf-mRNA-based classifiers that can distinguish AD patients from NCI individuals. To ensure that classifier performance is assessed unbiasedly, the cohort was first randomly split into a training set (65% of the cohort) and a testing set (35% of the cohort) (FIG. 6A). Subsequently, differential expression analysis was performed on the training set and all of the differentially expressed genes (1,476 genes, FDR<0.05) were selected as input features. Classification models were trained using the following algorithms: logistic regression with L1 regularization (LASSO), random forest, logistic regression with L2 regularization (ridge classifier), nearest neighbor classifier, and support vector machine (SVM) (FIG. 6B). Models trained on the training dataset were then applied to the testing set and their performance was evaluated by calculating AUROC (Area Under the Receiver Operating Characteristics). Of all the algorithms evaluated, ridge classifier provided the optimal classification performance with an AUROC of 0.902 (FIG. 6C), and a mean AUROC of 0.844 (FIG. 6D). Using the disease risk score cutoff of 0.44, the classifier had a sensitivity of 0.81 and a specificity of 0.85. By tuning the regularization parameter in the LASSO logistic regression classifiers, the number of features that are incorporated into the classifier was reduced. After incorporating varying numbers of genes in the classifier and assessing their performance on the testing set, the number of genes used in the classifier was reduced to 9 while maintaining high classification performance (AUROC=0.861). The expression of each of 9 genes (KIAA0100, MAGI1, NNMT, MXD1, ZNF75A, SELL, ASS1, MNDA, and AC132217.4 (non-coding RNA)) in the total patient cohort is shown in FIG. 6E.

### Example 9—Identification of Cf-mRNA Signatures that Correlate with AD Severity

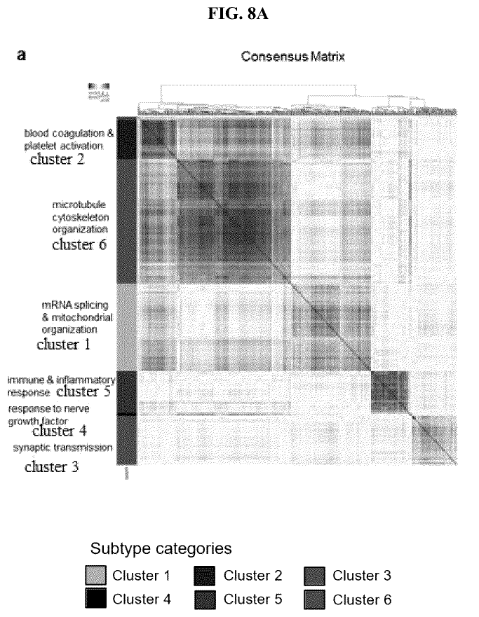

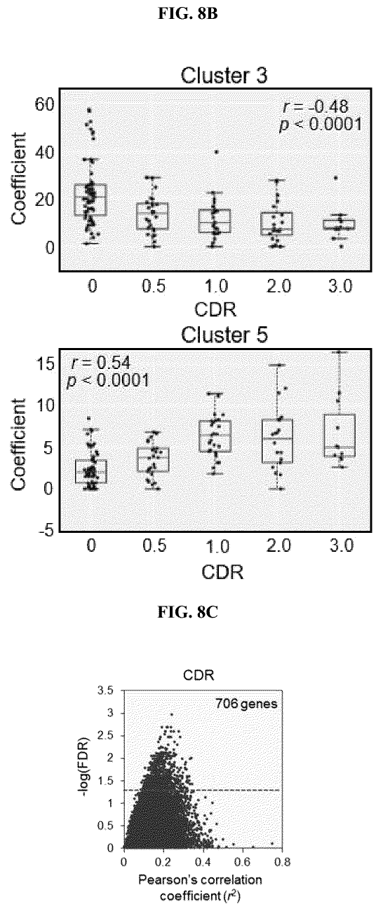

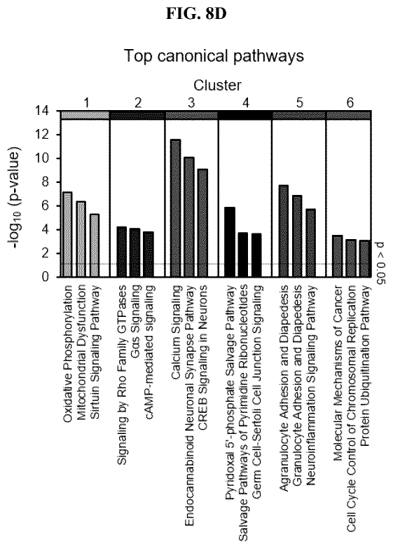

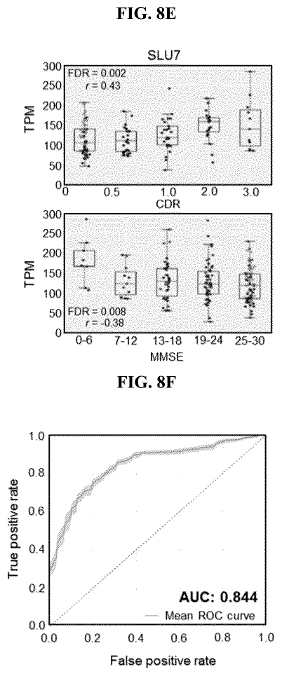

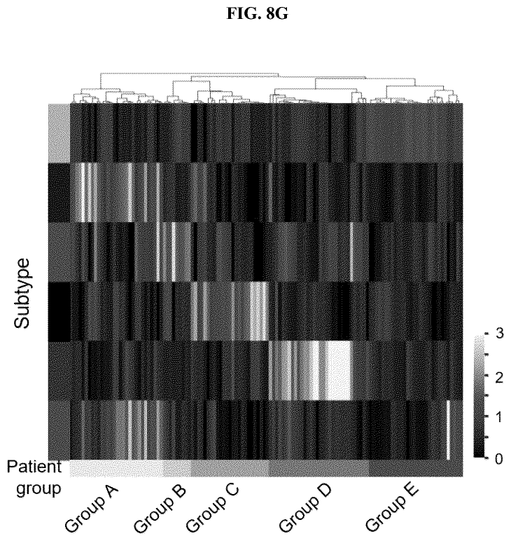

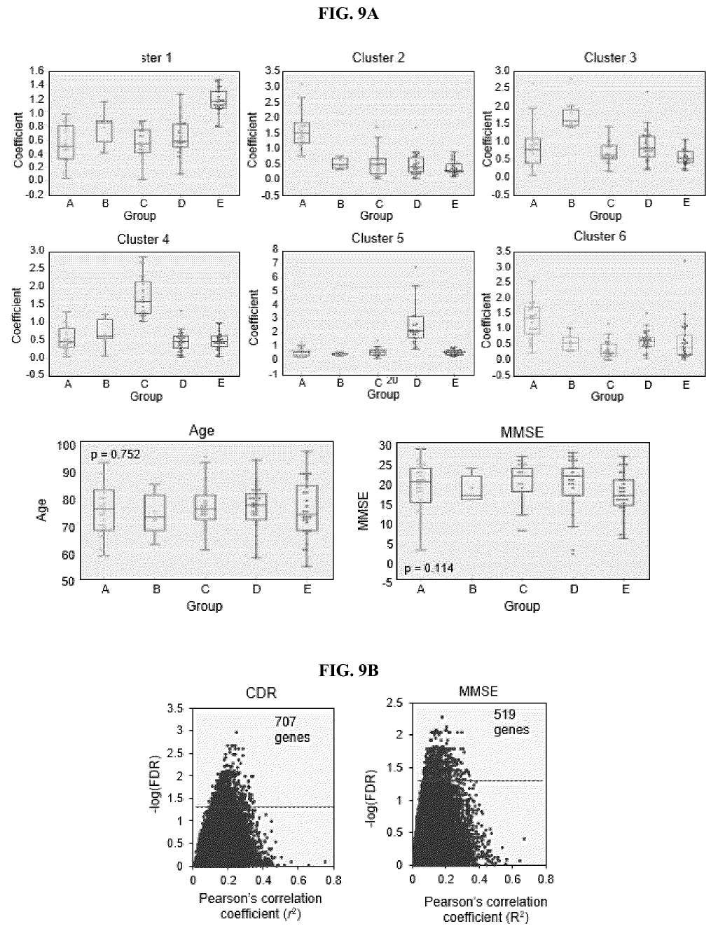

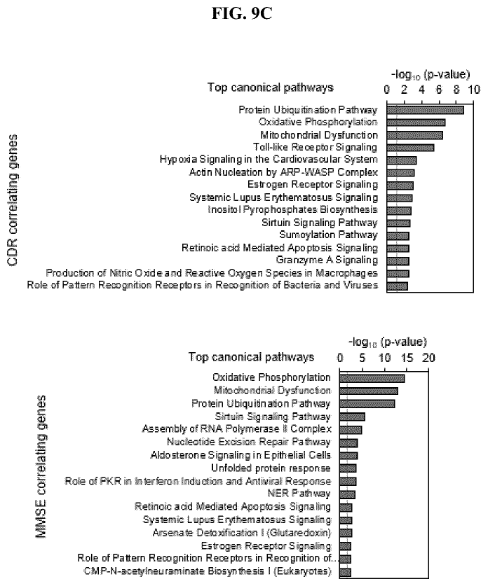

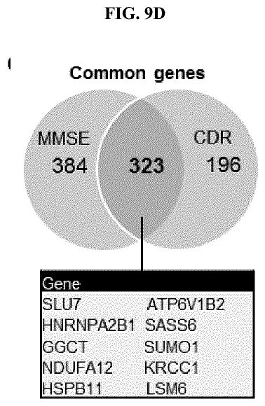

Unsupervised clustering on herein disclosed genes using non-negative matrix factorization (NMF) was used to identify six clusters of genes that were associated with distinct biological processes (FIGS. 8A and 9A).

The normalized expression values of two clusters, synaptic transmission, and immune and inflammatory response showed significant correlation with the CDR score (FIGS. 8B and 9A). Synaptic transmission cluster genes showed decreased expression with increasing CDR scores (r=−0.48, p<0.0001), and between CDR score 0 and 0.5 (p=0.001). In contrast, the expression levels of immune and inflammatory response cluster increased with CDR score up to 1, but the expression values did not increase for patients with higher CDR (r=0.54, p<0.0001).

Unsupervised decomposition using non-negative matrix factorization (NMF) identified six clusters of genes (FIG. 8A). IPA pathway analyses revealed association with processes involved in AD onset and progression, (FIG. 8A). For instance, Cluster 3 is enriched in genes associated with synaptic transmission pathways, while Cluster 5 is enriched in genes associated with immune response and neuroinflammation (FIG. 8A). A heterogeneous AD patient population was be stratified into subtypes based on the molecular profiles of these six gene-clusters. In particular, unsupervised hierarchical clustering of all 126 AD patients based on the magnitudes of the six gene clusters revealed five distinct groups (FIG. 8G). For example, “Group D” patients are characterized by elevated levels of Cluster 5 genes (e.g., immune response and neuroinflammation). The observed patient grouping was not due to sample source, age differences or the severity of cognitive impairment (FIG. 9A) and suggests that cf-mRNA profiling could be used for non-invasive subtyping of AD patients.

Next, in order to better understand the relationship between changes in these pathways/processes and the progression of AD, it was investigated whether any of these clusters correlate with the patient Clinical Dementia Rating (CDR) scores. The analysis revealed that the normalized expression values of two clusters of genes, Clusters 3 (“synaptic transmission”) and 5 (“immune response, neuroinflammation”) significantly correlated with the CDR score (FIG. 3D). In particular, “synaptic transmission” gene-cluster showed decreased expression with increasing CDR scores (r=−0.48, p value of correlation p<0.0001), and significant differences were observed even between individuals with no dementia (CDR=0) and patients with very mild dementia (CDR=0.5) (p=0.001). In contrast, the expression levels of “immune response and neuroinflammation” cluster increased with CDR score (r=0.54, p value of correlation p<0.0001), with most acute changes happening between CDR stages 0 to 1.

Based on these observations, individual genes whose expression levels significantly correlate with disease severity were sought. 707 genes were identified that correlated with CDR score (FDR<0.05, FIG. 9B). Gene ontology analyses revealed that these genes are primarily involved in proteostasis, oxidative phosphorylation and mitochondrial dysfunction (FIG. 9C), all well-known to be related to AD. To ensure that the genes correlate with cognitive impairment consistently, the same analysis was repeated using MMSE score, another widely used clinical metric for cognitive impairment assessment. 519 genes correlated with MMSE score (FIG. 9B). Genes identified correlating with CDR and MMSE scores overlapped significantly, as well as the molecular pathways identified using these genes (FIGS. 9C and 9D). Intriguingly, SLU7, a gene involved in pre-mRNA splicing, which has been shown to be dysregulated in the brain tissues of aging individuals and patients with neurodegenerative disorders (26), highly correlated with both CDR and MMSE scores (FIGS. 9D and 8E).

### Example 10—Aging Study Based on cfRNA

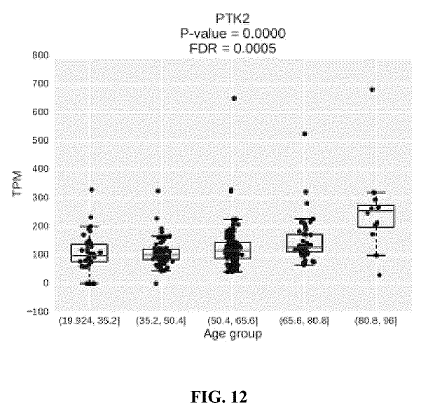

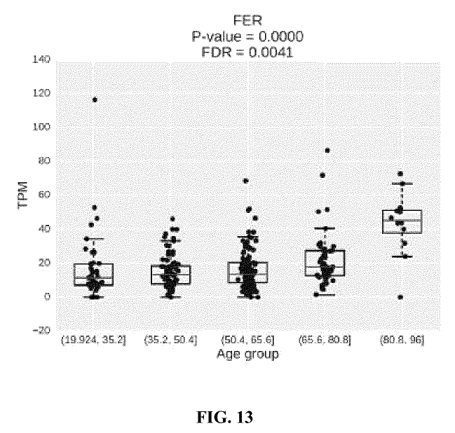

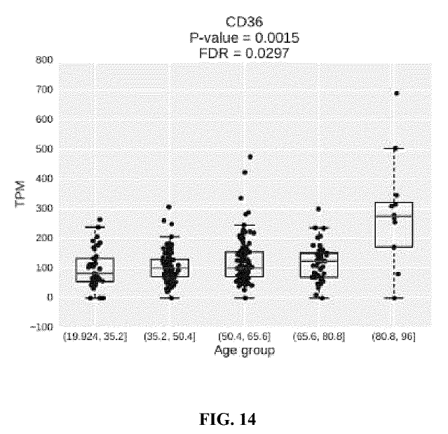

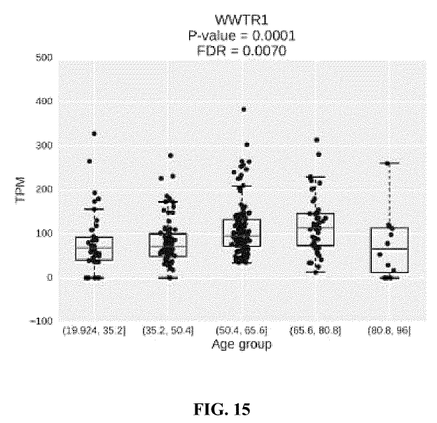

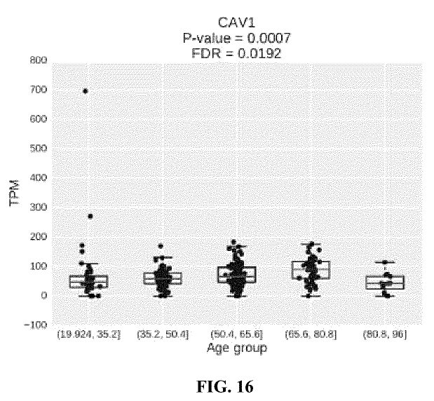

Gene expression, cfRNA data was gathered for 294 individuals who each had cfRNA expression data measured previously. The age of the subject at which the expression data was gathered was noted and the data was split into five bins based on chronological age ranges: 20-35, 35-50, 50-66, 66-81, and 81-96. A spearman correlation was calculated between the expression data and the age of the individual. A false discovery rate (FDR) cutoff of 0.05 was applied, yielding 774 genes found to be correlated with age. Of these 774 genes, 660 were positively correlated with age (upregulated), and 114 were negatively correlated with age (downregulated). FIGS. 11-16 show the differential expression of six genes found to be correlated with age: TCF7, PTK2, FER, CD36, WWTR1 and CAV1.

### Example 11—Relationship of Gene Expression with Proteins Reactive to Oxygen Species

The 774 genes identified in Example 10 were compared to gene set G00000302 “response to reactive oxygen species.” Interestingly, 18 of these genes correlated with age. This overlap between gene sets if significantly higher than would be expected by chance alone with a p-value of 4.99e-

### Example 12—Non-Blood Genes that Correlate with Age

Whole blood, buffy coat, and cf-RNA alone were sequenced for three individuals. Out of the 512 non-blood genes sequenced, meaning that they are only found in the cf-RNA fraction, 40 showed a correlation with age by the method used in Example 10.

### Example 13—Comparison of Age-Associated Genes with Other Datasets

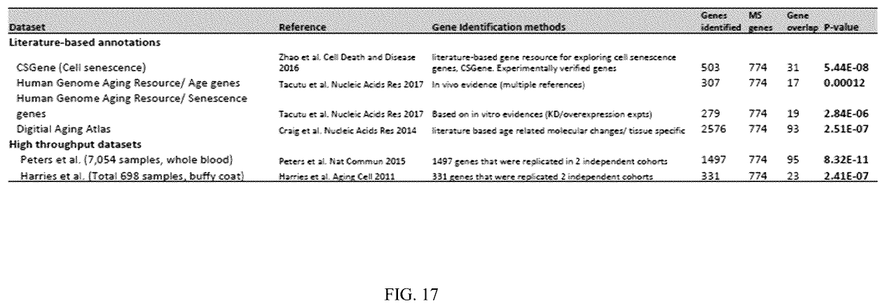

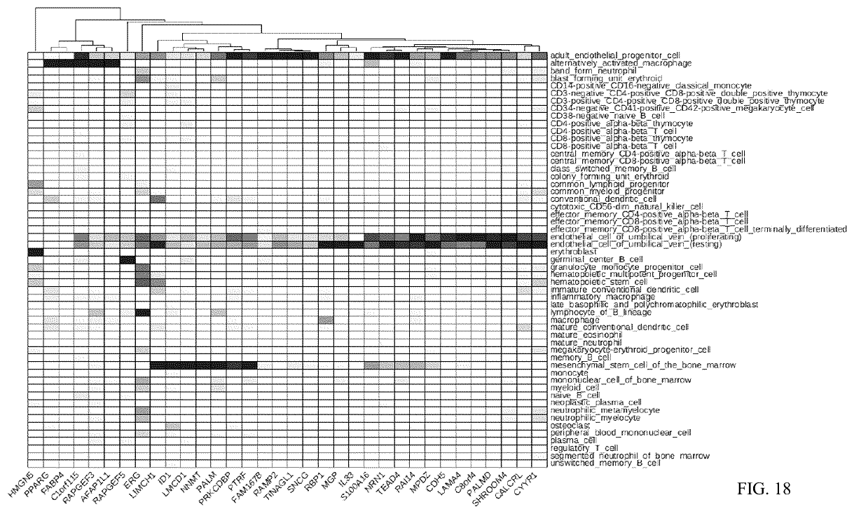

The 774 age-associated genes identified in Example 10 were compared with age-associated genes identified in other data sets, summarized in FIG. 17. The 774 genes identified overlap well with the gene sets, even prior to adjustment for confounding clinical parameters. Two genes, NELL2 and TLB are consistently highly correlated with age in all datasets in that they are in the top 30 differentially expressed genes in all datasets.

### Example 14—Correction for Confounder Effects

A multivariate regression was applied to correct the effects of confounders on the expression data gathered for the 774 age-associated genes. Exemplary confounders include but are not limited to pre-processing protocols (spins, filtering, etc.), types of bio fluid (serum v. plasma), and source of the sample (which center/university/hospital). The result of the regression was that 120 genes were significantly associated with age (FDR<0.1).

Of the 120 genes associated with age, 15 genes showed an expression decrease with age. These 15 genes include: LEF1, TCF7, and BCL11B.

Of the 120 genes associated with age, 105 genes showed an expression increase with age. These 105 genes include: ID1, CDKN1C, CDHS, and PPARG.

### Example 15—Overlap of 120 Genes Displaying Increased Expression with Age and Non-Blood Genes

Out of the 120 genes from Example 14 showing a correlation with age, 41 overlapped with the non-blood genes sequenced in Example 12. FIG. 18 shows a heat-map of the 41 genes. The p-value of the relationship is 3.93e-11. The 41 genes include HMGN5, PPARG, FABP4, C1orf115, RAPGEF3, AFAP1L1, RAPGEF5, ERG, LIMCH1, ID1, LMCD1, NNMT, PALM, PRKCDBP, PTRF, FAM167B, RAMP2, TINAGL1, SNCG, RBPi, MGP, IL33, S100A16, NRN1, TEAD4, RAI14, MPDZ, CDHS, LAMA4, C8orf4, PALMD, SHROOM4, CALCRL, and CYYR1.

### Example 16—Overlap of Age-Associated Genes with GTEx Data

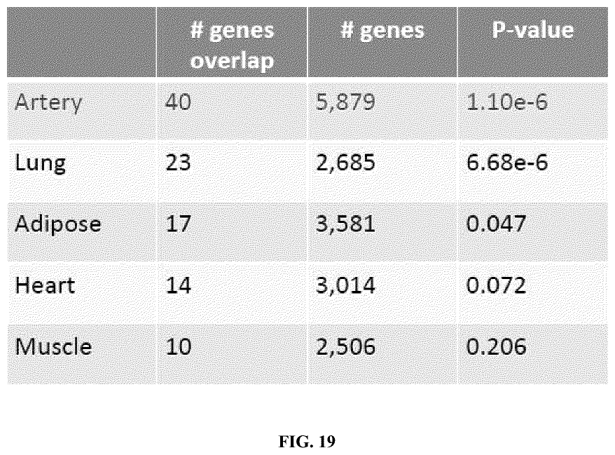

The 120 genes from Example 15 were compared to age-associated genes from GTEx data reported by Yang et al. summarized in FIG. 19.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-68. (canceled)

69. A method of detecting a risk of Alzheimer's disease (AD) in a subject, the method comprising:
(a) quantifying cell-free messenger RNA (cf-mRNA) levels of a plurality of cf-mRNAs in a biological sample; and
(b) processing one or more of said levels of said plurality of cf-mRNAs to identify a disease state of a tissue of said subject and an age of said subject,
wherein the processing comprises comparing the cf-mRNA levels in the subject to a threshold value of the plurality of cf-mRNAs.

70. The method of claim 69, wherein the biological sample comprises blood of the subject.

71. The method of claim 69, wherein the processing further comprises applying a machine learning classifier to said one or more of said levels of said plurality of cf-mRNAs.

72. The method of claim 71, wherein said machine learning classifier comprises a LASSO regression model.

73. The method of claim 69, further comprising (c) quantifying cf-mRNA levels of the plurality of cf-mRNAs in a second biological sample and (d) processing one or more of said levels of the plurality of cf-mRNAs in the second biological sample to identify a second disease state of said tissue of said subject.

74. The method of claim 73, wherein the second biological sample is obtained after the subject has received a treatment or therapy for a neurodegenerative disorder.

75. The method of claim 74, wherein the treatment or therapy comprises one or more of a cholinesterase inhibitors or memantine.

76. The method of claim 69, wherein the quantifying further comprises subjecting the plurality of cf-mRNAs to at least one of reverse transcription, polynucleotide amplification, sequencing, probe hybridization, microarray hybridization, or a combination thereof.

77. The method of claim 69, further comprising forming a next-generation sequencing (NGS) library comprising a plurality of cDNAs derived from the plurality of cf-mRNAs.

78. The method of claim 69, wherein the quantifying further comprises detecting a proportion of the plurality of cf-mRNA that contributes to the biological sample not from blood.

79. The method of claim 69, wherein the quantifying further comprises detecting a proportion of the plurality of cf-mRNAs that contributes to the biological sample from the subject's brain.

80. The method of claim 69, wherein the plurality of cf-mRNAs correspond to two or more genes selected from the group consisting of KIAA0lO0, MAGl1, NNMT, MXDl, ZNF75A, SELL, ASSl, MNDA, and AC132217.4.

81. The method of claim 69, further comprising identifying the subject as having a high risk of Alzheimer's disease and recommending a treatment for the subject.

82. The method of claim 81, further comprising treating the subject for Alzheimer's disease.

83. The method of claim 82, wherein said treatment comprises one or more of a cholinesterase inhibitors or memantine.

84. A method of detecting a risk of a stage of Alzheimer's disease (AD) in a subject, the method comprising:
(a) obtaining a biological sample from the subject; and
(b) detecting cell-free messenger RNA (cf-mRNA) levels of a plurality of cf-mRNAs in the biological sample, wherein the plurality of cf-mRNAs correspond to two or more genes selected from the group consisting of KIAA0lO0, MAGll, NNMT, MXD1, ZNF75A, SELL, ASSl, MNDA, and AC132217.4.

85. The method of claim 84, further comprising processing the levels of the plurality of cf-mRNAs using a machine learning classifier.

86. The method of claim 85, wherein the machine learning classifier comprises a LASSO regression model.

87. A method of detecting a risk of Alzheimer's disease (AD) in a subject, the method comprising:
(a) quantifying cell-free messenger RNA (cf-mRNA) levels of a plurality of cf-mRNAs in a biological sample, wherein the plurality of cell-free mRNA corresponds to genes encoding transcriptional factors involved in at least one of the sirtuin signaling pathway, IL-8 signaling pathway, protein ubiquitination pathway, oxidative phosphorylation pathway, sumoylation pathway, mitochondrial dysfunction pathway, inflammasome pathway, GABA receptor signaling pathway, netrin signaling pathway, synaptic long term depression signaling pathway, opioid signaling pathway, or a combination thereof; and
(b) comparing the cf-mRNA levels in the subject to a threshold value of the plurality of cf-mRNAs.
